(19) United States
(12) Patent Application Publication
(10) Pub. No.: US 2025/0171715 A1
(43) Pub. Date: May 29, 2025

(54) DETERGENT AND CLEANING AGENT CONTAINING ANTIMICROBIAL PEPTIDE

(71) Applicant: HENKEL AG & CO. KGAA, Düsseldorf (DE)

(72) Inventors: Stefan Evers, Haan (DE); Shohana Islam, Alsbach-Hähnlein (DE); Jan Schages, Düsseldorf (DE); Mirko Weide, Düsseldorf (DE); Christian Degering, Erkrath (DE); Susanne Wieland, Zons/Dormagen (DE); Iva Anic, Düsseldorf (DE); Irmgard Schmidt, Solingen (DE); Alexander Boes, Köln (DE); Felix Jakob, Erkelenz (DE); Ulrich Schwaneberg, Kelmis-Hergenrath (BE); Elisabeth Heine, Nideggen (DE)

(21) Appl. No.: 18/951,288

(22) Filed: Nov. 18, 2024

(30) Foreign Application Priority Data
Nov. 24, 2023 (DE) 102023211746.0

(51) Int. Cl. C11D 3/37; A01N 63/50; A01P 1/00; C07K 14/00; C11D 1/00; C11D 1/22; C11D 1/29; C11D 1/66; C11D 1/72; C11D 3/08; C11D 3/10; C11D 3/20; C11D 3/32; C11D 3/33; C11D 3/36; C11D 3/386; C11D 3/48; C11D 9/02; C11D 10/04

(52) U.S. Cl. CPC C11D 3/3719; A01N 63/50; A01P 1/00; C07K 14/00; C11D 3/08; C11D 3/10; C11D 3/2086; C11D 3/32; C11D 3/33; C11D 3/361; C11D 3/364; C11D 3/386; C11D 3/48; C11D 10/045; C11D 1/008; C11D 1/22; C11D 1/29; C11D 1/662; C11D 1/72; C11D 9/02

(57) ABSTRACT

The invention relates to the field of detergents and cleaning agents, in particular peptide-containing detergents and cleaning agents, and relates to the use of a peptide, in particular an antimicrobially active peptide, in a detergent and cleaning agent for cleaning textiles and/or hard surfaces, in particular dishes, and/or household surfaces. The detergent and cleaning agent according to the invention preferably comprises an antimicrobially active peptide, at least one surfactant, a builder system, and optionally at least one enzyme. Furthermore, the invention relates to corresponding agents and methods.

The invention relates to the field of detergents and cleaning agents, in particular peptide-containing detergents and cleaning agents, and relates to the use of a peptide, in particular an antimicrobially active peptide, in a detergent and cleaning agent for cleaning textiles and/or hard surfaces, in particular dishes, and/or household surfaces. Furthermore, the invention relates to corresponding agents and methods.

The most important criterion in cleaning textiles and/or hard surfaces is the cleaning performance on a wide variety of stains. Even though the cleaning performance of the detergents and cleaning agents used today is generally high, the general trend towards the increased use of low-temperature programs and the trend towards compacting formulations (in particular for pre-portioned forms of use) has led to the problem that many of the usual detergent and cleaning agent formulations have insufficient cleaning performance, in particular on stubborn stains. Such inadequate cleaning performance leads to consumer dissatisfaction and to such stains being pre-treated by the consumer, which in turn increases water and energy consumption.

Due to the trend towards low-temperature programs, microorganisms such as bacteria, fungi and viruses present on textiles and/or hard surfaces, in particular dishes, and/or household surfaces, preferably in washing machines and dishwashers, are not sufficiently killed and/or removed, so that this can lead to the spread of germs from one surface to another surface, e.g., from one textile to another textile during the washing or cleaning process. This can potentially pose a hygiene problem. This situation is exacerbated if bleach-free detergents, e.g., color detergents or liquid detergents or mild detergents, are used. This also applies to liquid machine and hand dishwashing detergents, which are also generally bleach-free and are used at rather low temperatures. The growth of microorganisms not only causes a hygiene problem, but can also lead to bad odors and/or microbial deposits on surfaces such as textiles, dishes and other household surfaces.

The term “antimicrobially active” or “antimicrobial activity”, as used herein, means that living microorganisms, such as bacteria, fungi or viruses, are put into a state in which they are inhibited in their growth or killed, or at least are no longer infectious or virulent. “Inhibited in growth” means that despite optimal growth conditions in a medium, the number of individual microorganisms capable of reproduction and/or viability remains substantially constant or decreases. “Killed” means that the number of reproducible and/or viable individual microorganisms in a medium is reduced compared to a reference value. Preferably, the antimicrobial activity, i.e., the reduction of viable and/or proliferative cells, is determined as described in Example 1 herein. According to the invention, antimicrobially active substances can comprise antibacterial as well as antifungal or antiviral substances, as well as bactericidal, bacteriostatic, virucidal, virustatic, fungicidal or fungistatic substances.

The term “bad odor” as used herein refers to an odor that is undesirable on textiles and/or hard surfaces, in particular dishes, and/or household surfaces. A wide variety of causes can cause an unpleasant odor, so-called bad odor, on surfaces, in particular household surfaces, such as surfaces in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets, as well as on washed textiles and/or cleaned hard surfaces, in particular dishes. This problem is known in particular for textiles and washing machines. But bad odors can also be found in the dishwasher. This affects all components that come into contact with dirty objects and/or water, in particular used water, and/or washing/cleaning suds, such as surfaces in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets. One, if not the most common, cause of this are organic in nature, such as sebum, body soils (e.g., dead skin cells and sweat), cell residues, food residues, body excretions, biofilms and extracellular polymeric substances (EPS; e.g., extracellular polymeric saccharides). In particular, microbially induced biofilms and bio-buildup, i.e., microbially induced bad odor as a result of microbially induced biofilms and bio-buildup, should also be emphasized here. Another example of unpleasant odors is sweat or body odor that clings to an object that has come into contact with people and/or animals. Bad odors are not only found in the washing machine, but also on the freshly washed, still damp laundry, as well as on the dry laundry, and on numerous other surfaces, in particular household surfaces that come into contact with soiled objects and/or water, in particular used water, and/or washing/cleaning suds, such as surfaces in washing machines or dishwashers, pump sumps, storage tanks, water reservoirs, washing drums, rinsing chambers, washing or dishwashing sinks, toilet bowls, toilet cisterns, etc. e.g., surfaces in washing machines or dishwashers, pump sumps, storage containers, water reservoirs, washing drums, rinsing chambers, washbasins or sinks, toilet bowls, cisterns, toilet rim, siphons, water pipes and/or lines, water inlets and/or outlets. However, biofilms, deposits, bio-buildups, body soils and the like not only lead to bad odors, but can also appear as stains on textiles and/or surfaces, in particular household surfaces, such as surfaces in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets, e.g., in the form of coatings, smears, incrustations, yellowing, armpit stains, graying or the like. Due to their typically adhesive texture, biofilms, bio-buildups and the like also allow other bad odors and/or dirt particles to adhere.

The term “biofilm” or “bio-buildup” as used herein is synonymous with the term “microbial deposit” and means any microbial deposit, biofilm, bio-buildup or the like that may be formed by any microorganism whose cells adhere to each other or to a surface, such as textiles, dishes or a hard surface or other type of surface. These adherent cells are often embedded in a self-produced matrix of extracellular polymeric substance (EPS). Biofilm EPS is a polymeric conglomerate generally comprising extracellular DNA, proteins and polysaccharides. Biofilms can form on living or non-living surfaces. The microbial cells that grow in a biofilm are physiologically different from the planktonic cells of the same organism, which, in contrast, are single cells that float or can float in a liquid medium. Bacteria living in a biofilm usually have significantly different properties than planktonic bacteria of the same species, because the dense and protected environment of the film allows them to cooperate and interact in different ways. One advantage of this environment for the microorganisms is the increased resistance to detergents and cleaning agents and antibiotics, as the dense extracellular matrix and the outer cell layer protect the inside of the community. Biofilm-producing bacteria on textiles include, for example, the following species: Acinetobacter sp., Aeromicrobium sp., Brevundimonas sp., Microbacterium sp., Micrococcus luteus, Pseudomonas sp., Staphylococcus epidermidis and Stenotrophomonas sp. On hard surfaces, biofilm-forming bacteria can be found, for example, among the following species: Acinetobacter sp., Aeromicrobium sp., Brevundimonas sp., Microbacterium sp., Micrococcus luteus, Pseudomonas sp, Staphylococcus epidermidis, Staphylococcus aureus and Stenotrophomonas sp.

Accumulated organic soiling is often difficult to remove, and commercially available detergents and cleaning agents do not sufficiently reduce the bad odor. Instead, the bad odor is masked by the use of various fragrances, which is often perceived by consumers as a cleaning effect, although the causes of the bad odors are not actually eliminated. As a result, more and more organic material and therefore more and more bad odors accumulate over time and more and more fragrances have to be added to prevent the textiles from smelling worse and worse. However, many fragrances have side effects such as allergenic properties, which can manifest themselves in the form of skin rashes. In addition, many of these substances, as natural chemicals, are harmful to the environment if they get into wastewater. In recent years, fragrances have increasingly been used in the form of fragrance microcapsules, although it is known that microcapsules are considered microplastics and are therefore potentially harmful to the environment. Fragrances are also expensive and add to the overall cost of detergents and cleaning products.

There is therefore a need to improve the cleaning performance of detergents and cleaning agents, in particular with regard to the elimination of bad odors in washing machines and dishwashers and/or on the washed textiles or cleaned surfaces, in particular dishes, as well as surfaces, in particular household surfaces, such as surfaces in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets. There is also a need to improve hygiene in washing machines and dishwashers and to reliably remove microbial biofilms/bio-buildups from surfaces, in particular household surfaces, such as surfaces in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets. This also includes (semi-)automated washing or cleaning systems such as floor-mopping robots or wet vacuum cleaners.

Surprisingly, it has now been found that peptides according to the invention are antimicrobially active and prevent and/or reduce the growth of microorganisms. They can therefore not only help to improve washing and cleaning performance in terms of hygiene, but also to prevent microbially-induced bad odors and microbially-induced deposits (biofilms, bio-buildups and the like).

Peptides according to the invention can also be antimicrobially active in washing and care products for hair care as well as in industrial and institutional cleaners such as disinfectant products, in particular disinfectants.

In a first aspect, it is therefore an object of the invention to provide an agent, preferably a detergent and cleaning agent, preferably a liquid detergent and cleaning agent, particularly preferably a liquid textile detergent, comprising
(A) at least one peptide, wherein the peptide
   has a length of 10 to 50, preferably 10 to 30, preferably 12 to 25, amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, preferably selected from alanine, arginine, glutamic acid, glycine, isoleucine, leucine, lysine, phenylalanine, proline, serine, threonine, tryptophan, tyrosine and valine,
   has a net charge of +2 to +8, preferably +3 to +7, preferably +4 to +6,
   is antimicrobially active, the antimicrobial activity being determined as described in Example 1,
(B) at least one surfactant, wherein the surfactant is selected from the group consisting of anionic surfactants, non-ionic surfactants, cationic surfactants, zwitterionic surfactants, amphoteric surfactants and mixtures thereof;
(C) a builder system comprising at least one builder, wherein the builder is selected from the group consisting of polycarboxylic acids such as citric acid, adipic acid, succinic acid, glutaric acid, malic acid, tartaric acid, maleic acid, fumaric acid, saccharic acids and carboxymethyl inulines or salts thereof, monomeric and polymeric aminopolycarboxylic acids such as glycine diacetic acid, methylglycine diacetic acid (MGDA), glutamic acid diacetic acid (GLDA), nitrile triacetic acid, iminodisuccinate such as ethylenediamine-N,N′-disuccinic acid and hydroxyiminodisuccinates, ethylenediaminetetraacetic acid and polyaspartic acid or salts thereof, polyphosphonic acids such as aminotris(methylenephosphonic acid), ethylenediaminetetrakis(methylenephosphonic acid), lysinetetra(methylenephosphonic acid), diethylenetriaminepenta(methylenephosphonic acid) (DTPMP) and 1-hydroxyethane-1,1-diphosphonic acid (HEDP) or salts thereof, polymeric hydroxy compounds such as dextrin, and mixtures thereof; and
(D) optionally at least one enzyme, wherein the enzyme is selected from the group consisting of proteases, amylases, lipases, mannanases, cellulases or mixtures thereof.

In a further aspect, the invention relates to an agent, preferably a detergent and cleaning agent, wherein the agent comprises
(A) at least one peptide, wherein the peptide
   has a length of 10 to 50, preferably 10 to 30, preferably 12 to 25, amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, preferably selected from alanine, arginine, glutamic acid, glycine, isoleucine, leucine, lysine, phenylalanine, proline, serine, threonine, tryptophan, tyrosine and valine,
   has a net charge of +2 to +8, preferably +3 to +7, preferably +4 to +6,
   is antimicrobially active, the antimicrobial activity being determined as described in Example 1, in an amount of active peptide from 1×10⁻⁸ to 5 wt. %, preferably 0.0001 to 1 wt. %, preferably 0.0005 to 0.5 wt. %, and particularly preferably 0.001 to 0.1 wt. %, based on the total weight of the agent;
(B) 2 to 20 wt. %, preferably 3 to 17 wt. %, of anionic surfactants,
(C) 1 to 10 wt. %, preferably 3 to 8 wt. %, of non-ionic surfactants,
(D) 0 to 1 wt. %, preferably 0 to 0.5 wt. %, of soap, and
(E) 0 to 5 wt. %, preferably 0 to 3 wt. %, of fatty acids,
in each case based on the total weight of the agent.

In still another aspect, the invention relates to an agent, preferably a detergent and cleaning agent, wherein the agent comprises
(A) at least one peptide, wherein the peptide
   has a length of 10 to 50, preferably 10 to 30, preferably 12 to 25, amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, preferably selected from alanine, arginine, glutamic acid, glycine, isoleucine, leucine, lysine, phenylalanine, proline, serine, threonine, tryptophan, tyrosine and valine,
   has a net charge of +2 to +8, preferably +3 to +7, preferably +4 to +6,
   is antimicrobially active, the antimicrobial activity being determined as described in Example 1,
   in an amount of active peptide from 1×10⁻⁸ to 5 wt. %, preferably 0.0001 to 1 wt. %, preferably 0.0005 to 0.5 wt. %, and particularly preferably 0.001 to 0.1 wt. %, based on the total weight of the agent;
(B) 0 to 10 wt. %, preferably 1 to 4 wt. %, of citric acid and/or citrate, preferably alkali citrate,
(C) 0 to 40 wt. %, preferably 0 to 15 wt. %, more preferably 1 to 3 wt. %, of alkali carbonate, preferably sodium carbonate,
(D) 0 to 20 wt. %, preferably 3 to 10 wt. %, of alkali silicate,
(E) 0 to 10 wt. %, preferably 0.5 to 2 wt. %, of phosphonic acid and/or alkali phosphonate, particularly preferably HEDP and/or DTPMP, and/or
(F) 0 to 10 wt. %, preferably 0.5 to 3 wt. %, of aminopolycarboxylic acids, preferably MGDA and/or GLDA,
   in each case based on the total weight of the agent.

In preferred embodiments, the peptide comprises at least 1 to 5 tryptophan functional groups and/or 1 to 5 arginine functional groups and/or 1 to 5 lysine functional groups, preferably at least 1 to 4 tryptophan functional groups and/or 1 to 3 arginine functional groups and/or 1 to 5 lysine functional groups.

In more preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequence set forth in SEQ ID NO:1, SEQ ID NO:2, SEQ ID NO:3, SEQ ID NO:4 or SEQ ID NO:5.

In preferred embodiments, the peptide according to the invention is contained in an agent, preferably a detergent and cleaning agent, wherein
the agent is substantially free of boron-containing compounds, preferably free of boron-containing compounds; and/or
the agent is substantially free of phosphonates, preferably free of phosphonates, in particular free of HEDP and/or DTPMP; and/or
the agent has a pH in a range of from about 6 to about 11, in particular from about 6.5 to about 10.5, more preferably from about 7 to about 10, particularly preferably from about 8 to about 9 in a 1 wt. % solution in deionized water at 20° C.; and/or
the agent contains, based on the total weight of the agent, less than 10 wt. % LAS, preferably less than 6 wt. %, more preferably less than 3 wt. %, even more preferably is substantially free of LAS, particularly preferably is free of LAS; and/or
the agent is substantially free of bleach, preferably free of bleach.

Further objects of the invention relate to
a method for cleaning textiles and/or hard surfaces, in particular dishes, characterized in that a detergent and cleaning agent described herein is used in at least one method step, the method preferably being carried out in a temperature range from about 20° C. to about 60° C., preferably about 40° C., particularly preferably about 30° C.;
a method for cleaning surfaces, in particular household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets, and/or textiles, in particular for preventing and/or reducing bad odors and/or for improving hygiene, characterized in that a detergent and cleaning agent described herein is used in at least one method step, the method preferably being carried out in a temperature range from about 20° C. to about 60° C., preferably about 40° C., particularly preferably about 30° C.;
a use of a peptide in a detergent and cleaning agent for cleaning textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets, preferably in a temperature range from about 20° C. to about 60° C., preferably about 40° C., particularly preferably about 30° C., the peptide being as defined herein;
a use of a peptide in a detergent and cleaning agent for reducing bad odors from textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets which have been treated with the agent, preferably in a temperature range from about 20° C. to about 60° C., preferably about 40° C., particularly preferably about 30° C., the peptide being as defined herein;
a use of a peptide in a detergent and cleaning agent for disinfecting textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets which have been treated with the agent, preferably in a temperature range from about 20° C. to about 60° C., preferably about 40° C., particularly preferably about 30° C., the peptide being as defined herein;
a use of a peptide in a detergent and cleaning agent for preventing and/or reducing microbial grown on textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets which have been treated with the agent, preferably in a temperature range from about 20° C. to about 60° C., preferably about 40° C., particularly preferably about 30° C., the peptide being as defined herein.

In a further aspect, the invention also relates to the use of a detergent and cleaning agent described herein for cleaning textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets, and/or for reducing bad odors from textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets, which have been treated with the agent, and/or for disinfecting textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets, which have been treated with the agent, and/or for preventing and/or reducing microbial growth on textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets, which have been treated with the agent, preferably in a temperature range from about 20° C. to about 60° C., preferably about 40° C., particularly preferably about 30° C.

These and other aspects, features and advantages of the invention will become apparent to a person skilled in the art through the study of the following detailed description and claims. Any feature from one aspect of the invention can be used in any other aspect of the invention. Furthermore, it will readily be understood that the examples contained herein are intended to describe and illustrate but not to limit the invention and that, in particular, the invention is not limited to these examples.

Unless indicated otherwise, all percentages are indicated in terms of weight percent (wt. %).

Numerical ranges that are indicated in the format “from x to y” also include the stated values. If several preferred numerical ranges are specified in this format, it is readily understood that any ranges resulting from the combination of the various endpoints are also included.

“At least one,” as used herein, means one or more, i.e., 1, 2, 3, 4, 5, 6, 7, 8, 9, 10, 11, 12, 13, 14 or more.

The term “detergent and cleaning agents” or “detergent or cleaning agent,” as used herein, is synonymous with the term “agent” and denotes a composition for cleaning textiles and/or hard surfaces, in particular dishes, as explained in the description. Also comprised are cleaning agents for cleaning surfaces, in particular household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets. This also comprises agents for (semi-)automated washing or cleaning systems such as floor-mopping robots or wet vacuum cleaners.

“Approximately,” “about,” or “roughly,” as used herein in reference to a numerical value, refers to the corresponding numerical value±10%, preferably ±5%.

“Liquid,” as used herein, includes liquids and gels as well as pasty compositions. It is preferred that the liquid compositions are flowable and pourable at room temperature, but it is also possible for them to have a limit of liquidity.

A substance, e.g., a composition or an agent, is solid according to the definition of the invention if it is in a solid state of aggregation at 25° C. and 1013 mbar.

A substance, e.g., a composition or an agent, is liquid according to the definition of the invention if it is in a liquid state of aggregation at 25° C. and 1013 mbar. Liquid also includes gel form.

“Variant,” as used herein refers to natural or artificially produced variations of a native enzyme or peptide having an amino acid sequence that is modified from the reference form.

The term “textile” as used herein means any textile material, including yarns, yarn intermediates, fibers, nonwovens, natural materials, synthetic materials and all other textile materials, fabrics made from these materials and products produced from fabrics (e.g., garments and other articles). The textile or fabric can be in the form of knitted fabrics, woven fabrics, denims, nonwovens, felts, yarns and terry cloth. The textile can be based on cellulose, e.g., natural cellulose fibers such as cotton, flax/linen, jute, ramie, sisal or coconut fibers, or artificially produced cellulose fibers (e.g., from cellulose pulp) such as viscose/rayon, cellulose acetate fibers (Tricell), Lyocell or blends thereof. The textile or fabric may also consist of non-cellulose fibers, e.g., natural polyamides such as wool, camel, cashmere, mohair, rabbit and silk, or synthetic polymers such as nylon, aramid, polyester, acrylic, polypropylene and spandex/elastane or blends thereof, as well as blends of cellulose fibers and non-cellulose fibers. Examples of blends are blends of cotton and/or rayon/viscose having one or more accompanying materials such as wool, synthetic fibers (e.g., polyamide fibers, acrylic fibers, polyester fibers, polyvinyl chloride fibers, polyurethane fibers, polyurea fibers, aramid fibers) and/or cellulose-containing fibers (e.g., rayon/viscose, ramie, flax/linen, jute, cellulose acetate fibers, Lyocell). The fabric can be conventional washable laundry, e.g., soiled household laundry. When the term “fabric” or “garment” is used, it should also comprise the broader term “textiles.”

The present invention is based on the surprising finding of the inventors that peptides according to the invention prevent and/or reduce the growth of microorganisms, such as bacteria, fungi or viruses, and/or reduce the number of viable and/or reproducible individual microorganisms.

According to the invention, a peptide which has a length of 10 to 50, preferably 10 to 30, preferably 12 to 25, amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, preferably selected from alanine, arginine, glutamic acid, glycine, isoleucine, leucine, lysine, phenylalanine, proline, serine, threonine, tryptophan, tyrosine and valine, has a net charge of +2 to +8, preferably +3 to +7, preferably +4 to +6, and is antimicrobially active, the antimicrobial activity being determined as described in Example 1, is used in a detergent and cleaning agent.

The use of a peptide according to the invention in detergent and cleaning agents or the use of a detergent and cleaning agent according to the invention leads to improved cleaning of textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets, and/or to a reduction of bad odors from textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets, and/or to the prevention and/or reduction of microbial growth on textiles and/or hard surfaces, in particular dishes, and/or household surfaces, and/or to the prevention and/or reduction of microbial growth on textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets.

This is particularly surprising, as the use of such peptides in detergents and cleaning agents has not yet been described. In addition, the effects described herein, which such peptides realize on textiles and/or hard surfaces, in particular dishes, and/or household surfaces, have also not been described, in particular that such peptides prevent and/or reduce the growth of microorganisms and/or help to hygienically improve the washing and cleaning performance, and/or prevent microbially induced bad odors as well as microbially induced deposits (biofilms, bio-buildups and the like).

According to the invention, a peptide is used which has a length of 10 to 50, preferably 10 to 30, preferably 12 to 25, amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, preferably selected from alanine, arginine, glutamic acid, glycine, isoleucine, leucine, lysine, phenylalanine, proline, serine, threonine, tryptophan, tyrosine and valine, has a net charge of +2 to +8, preferably +3 to +7, preferably +4 to +6, and is antimicrobially active, the antimicrobial activity being determined as described in Example 1.

In preferred embodiments, the peptide comprises at least 1 to 5 tryptophan functional groups and/or 1 to 5 arginine functional groups and/or 1 to 5 lysine functional groups, preferably at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the amino acid sequence set forth in SEQ ID NO:1, SEQ ID NO:2, SEQ ID NO:3, SEQ ID NO:4 or SEQ ID NO:5.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50, preferably 10 to 30, preferably 12 to 25, amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, preferably selected from alanine, arginine, glutamic acid, glycine, isoleucine, leucine, lysine, phenylalanine, proline, serine, threonine, tryptophan, tyrosine and valine, has a net charge of +2 to +8, preferably +3 to +7, preferably +4 to +6, and is antimicrobially active, the antimicrobial activity being determined as described in Example 1.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50 amino acids selected from the group consisting of alanine, arginine, glutamic acid, glycine, isoleucine, leucine, lysine, phenylalanine, proline, serine, threonine, tryptophan, tyrosine and valine, has a net charge of +2 to +8, and is antimicrobially active, the antimicrobial activity being determined as described in Example 1.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50 amino acids selected from the group consisting of alanine, arginine, glutamic acid, glycine, isoleucine, leucine, lysine, phenylalanine, proline, serine, threonine, tryptophan, tyrosine and valine, has a net charge of +3 to +7, and is antimicrobially active, the antimicrobial activity being determined as described in Example 1.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50 amino acids selected from the group consisting of alanine, arginine, glutamic acid, glycine, isoleucine, leucine, lysine, phenylalanine, proline, serine, threonine, tryptophan, tyrosine and valine, has a net charge of +4 to +6, and is antimicrobially active, the antimicrobial activity being determined as described in Example 1.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 30 amino acids selected from the group consisting of alanine, arginine, glutamic acid, glycine, isoleucine, leucine, lysine, phenylalanine, proline, serine, threonine, tryptophan, tyrosine and valine, has a net charge of +2 to +8, and is antimicrobially active, the antimicrobial activity being determined as described in Example 1.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 30 amino acids selected from the group consisting of alanine, arginine, glutamic acid, glycine, isoleucine, leucine, lysine, phenylalanine, proline, serine, threonine, tryptophan, tyrosine and valine, has a net charge of +3 to +7, and is antimicrobially active, the antimicrobial activity being determined as described in Example 1.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 30 amino acids selected from the group consisting of alanine, arginine, glutamic acid, glycine, isoleucine, leucine, lysine, phenylalanine, proline, serine, threonine, tryptophan, tyrosine and valine, has a net charge of +4 to +6, and is antimicrobially active, the antimicrobial activity being determined as described in Example 1.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 12 to 25 amino acids selected from the group consisting of alanine, arginine, glutamic acid, glycine, isoleucine, leucine, lysine, phenylalanine, proline, serine, threonine, tryptophan, tyrosine and valine, has a net charge of +2 to +8, and is antimicrobially active, the antimicrobial activity being determined as described in Example 1.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 12 to 25 amino acids selected from the group consisting of alanine, arginine, glutamic acid, glycine, isoleucine, leucine, lysine, phenylalanine, proline, serine, threonine, tryptophan, tyrosine and valine, has a net charge of +3 to +7, and is antimicrobially active, the antimicrobial activity being determined as described in Example 1.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 12 to 25 amino acids selected from the group consisting of alanine, arginine, glutamic acid, glycine, isoleucine, leucine, lysine, phenylalanine, proline, serine, threonine, tryptophan, tyrosine and valine, has a net charge of +4 to +6, and is antimicrobially active, the antimicrobial activity being determined as described in Example 1.

In preferred embodiments, the peptide comprises an amino acid sequence that, over the entire length thereof, is at least 70%, and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%, identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50, preferably 10 to 30, preferably 12 to 25, amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, preferably selected from alanine, arginine, glutamic acid, glycine, isoleucine, leucine, lysine, phenylalanine, proline, serine, threonine, tryptophan, tyrosine and valine, has a net charge of +2 to +8, preferably +3 to +7, preferably +4 to +6, and is antimicrobially active, the antimicrobial activity being determined as described in Example 1, wherein the peptide comprises at least 1 to 5 tryptophan functional groups and/or 1 to 5 arginine functional groups and/or 1 to 5 lysine functional groups, preferably at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50 amino acids selected from the group consisting of alanine, arginine, glutamic acid, glycine, isoleucine, leucine, lysine, phenylalanine, proline, serine, threonine, tryptophan, tyrosine and valine, has a net charge of +2 to +8, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50 amino acids selected from the group consisting of alanine, arginine, glutamic acid, glycine, isoleucine, leucine, lysine, phenylalanine, proline, serine, threonine, tryptophan, tyrosine and valine, has a net charge of +3 to +7, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50 amino acids selected from the group consisting of alanine, arginine, glutamic acid, glycine, isoleucine, leucine, lysine, phenylalanine, proline, serine, threonine, tryptophan, tyrosine and valine, has a net charge of +4 to +6, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 30 amino acids selected from the group consisting of alanine, arginine, glutamic acid, glycine, isoleucine, leucine, lysine, phenylalanine, proline, serine, threonine, tryptophan, tyrosine and valine, has a net charge of +2 to +8, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 30 amino acids selected from the group consisting of alanine, arginine, glutamic acid, glycine, isoleucine, leucine, lysine, phenylalanine, proline, serine, threonine, tryptophan, tyrosine and valine, has a net charge of +3 to +7, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 30 amino acids selected from the group consisting of alanine, arginine, glutamic acid, glycine, isoleucine, leucine, lysine, phenylalanine, proline, serine, threonine, tryptophan, tyrosine and valine, has a net charge of +4 to +6, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 12 to 25 amino acids selected from the group consisting of alanine, arginine, glutamic acid, glycine, isoleucine, leucine, lysine, phenylalanine, proline, serine, threonine, tryptophan, tyrosine and valine, has a net charge of +2 to +8, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 12 to 25 amino acids selected from the group consisting of alanine, arginine, glutamic acid, glycine, isoleucine, leucine, lysine, phenylalanine, proline, serine, threonine, tryptophan, tyrosine and valine, has a net charge of +3 to +7, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 12 to 25 amino acids selected from the group consisting of alanine, arginine, glutamic acid, glycine, isoleucine, leucine, lysine, phenylalanine, proline, serine, threonine, tryptophan, tyrosine and valine, has a net charge of +4 to +6, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to the amino acid sequence set forth in SEQ ID NO:1, SEQ ID NO:2, SEQ ID NO:3, SEQ ID NO:4 or SEQ ID NO:5.

In preferred embodiments, the peptide comprises an amino acid sequence that, over the entire length thereof, is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50, preferably 10 to 30, preferably 12 to 25, amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, preferably selected from alanine, arginine, glutamic acid, glycine, isoleucine, leucine, lysine, phenylalanine, proline, serine, threonine, tryptophan, tyrosine and valine, has a net charge of +2 to +8, preferably +3 to +7, preferably +4 to +6, and is antimicrobially active, the antimicrobial activity being determined as described in Example 1.

In preferred embodiments, the peptide comprises an amino acid sequence that, over the entire length thereof, is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50 amino acids selected from the group consisting of alanine, arginine, glutamic acid, glycine, isoleucine, leucine, lysine, phenylalanine, proline, serine, threonine, tryptophan, tyrosine and valine, has a net charge of +2 to +8, and is antimicrobially active, the antimicrobial activity being determined as described in Example 1.

In preferred embodiments, the peptide comprises an amino acid sequence that, over the entire length thereof, is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50 amino acids selected from the group consisting of alanine, arginine, glutamic acid, glycine, isoleucine, leucine, lysine, phenylalanine, proline, serine, threonine, tryptophan, tyrosine and valine, has a net charge of +3 to +7, and is antimicrobially active, the antimicrobial activity being determined as described in Example 1.

In preferred embodiments, the peptide comprises an amino acid sequence that, over the entire length thereof, is at least at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50 amino acids selected from the group consisting of alanine, arginine, glutamic acid, glycine, isoleucine, leucine, lysine, phenylalanine, proline, serine, threonine, tryptophan, tyrosine and valine, has a net charge of +4 to +6, and is antimicrobially active, the antimicrobial activity being determined as described in Example 1.

In preferred embodiments, the peptide comprises an amino acid sequence that, over the entire length thereof, is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 30 amino acids selected from the group consisting of alanine, arginine, glutamic acid, glycine, isoleucine, leucine, lysine, phenylalanine, proline, serine, threonine, tryptophan, tyrosine and valine, has a net charge of +2 to +8, and is antimicrobially active, the antimicrobial activity being determined as described in Example 1.

In preferred embodiments, the peptide comprises an amino acid sequence that, over the entire length thereof, is at least at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 30 amino acids selected from the group consisting of alanine, arginine, glutamic acid, glycine, isoleucine, leucine, lysine, phenylalanine, proline, serine, threonine, tryptophan, tyrosine and valine, has a net charge of +3 to +7, and is antimicrobially active, the antimicrobial activity being determined as described in Example 1.

In preferred embodiments, the peptide comprises an amino acid sequence that, over the entire length thereof, is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 30 amino acids selected from the group consisting of alanine, arginine, glutamic acid, glycine, isoleucine, leucine, lysine, phenylalanine, proline, serine, threonine, tryptophan, tyrosine and valine, has a net charge of +4 to +6, and is antimicrobially active, the antimicrobial activity being determined as described in Example 1.

In preferred embodiments, the peptide comprises an amino acid sequence that, over the entire length thereof, is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 12 to 25 amino acids selected from the group consisting of alanine, arginine, glutamic acid, glycine, isoleucine, leucine, lysine, phenylalanine, proline, serine, threonine, tryptophan, tyrosine and valine, has a net charge of +2 to +8, and is antimicrobially active, the antimicrobial activity being determined as described in Example 1.

In preferred embodiments, the peptide comprises an amino acid sequence that, over the entire length thereof, is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 12 to 25 amino acids selected from the group consisting of alanine, arginine, glutamic acid, glycine, isoleucine, leucine, lysine, phenylalanine, proline, serine, threonine, tryptophan, tyrosine and valine, has a net charge of +3 to +7, and is antimicrobially active, the antimicrobial activity being determined as described in Example 1.

In preferred embodiments, the peptide comprises an amino acid sequence that, over the entire length thereof, is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 12 to 25 amino acids selected from the group consisting of alanine, arginine, glutamic acid, glycine, isoleucine, leucine, lysine, phenylalanine, proline, serine, threonine, tryptophan, tyrosine and valine, has a net charge of +4 to +6, and is antimicrobially active, the antimicrobial activity being determined as described in Example 1.

In preferred embodiments, the peptide comprises an amino acid sequence that, over the entire length thereof, is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50, preferably 10 to 30, preferably 12 to 25, amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, preferably selected from alanine, arginine, glutamic acid, glycine, isoleucine, leucine, lysine, phenylalanine, proline, serine, threonine, tryptophan, tyrosine and valine, has a net charge of +2 to +8, preferably +3 to +7, preferably +4 to +6, and is antimicrobially active, the antimicrobial activity being determined as described in Example 1, wherein the peptide comprises at least 1 to 5 tryptophan functional groups and/or 1 to 5 arginine functional groups and/or 1 to 5 lysine functional groups, preferably at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50 amino acids selected from the group consisting of alanine, arginine, glutamic acid, glycine, isoleucine, leucine, lysine, phenylalanine, proline, serine, threonine, tryptophan, tyrosine and valine, has a net charge of +2 to +8, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50 amino acids selected from the group consisting of alanine, arginine, glutamic acid, glycine, isoleucine, leucine, lysine, phenylalanine, proline, serine, threonine, tryptophan, tyrosine and valine, has a net charge of +3 to +7, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50 amino acids selected from the group consisting of alanine, arginine, glutamic acid, glycine, isoleucine, leucine, lysine, phenylalanine, proline, serine, threonine, tryptophan, tyrosine and valine, has a net charge of +4 to +6, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 30 amino acids selected from the group consisting of alanine, arginine, glutamic acid, glycine, isoleucine, leucine, lysine, phenylalanine, proline, serine, threonine, tryptophan, tyrosine and valine, has a net charge of +2 to +8, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 80% and increasingly preferably at least 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 30 amino acids selected from the group consisting of alanine, arginine, glutamic acid, glycine, isoleucine, leucine, lysine, phenylalanine, proline, serine, threonine, tryptophan, tyrosine and valine, has a net charge of +3 to +7, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 30 amino acids selected from the group consisting of alanine, arginine, glutamic acid, glycine, isoleucine, leucine, lysine, phenylalanine, proline, serine, threonine, tryptophan, tyrosine and valine, has a net charge of +4 to +6, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 12 to 25 amino acids selected from the group consisting of alanine, arginine, glutamic acid, glycine, isoleucine, leucine, lysine, phenylalanine, proline, serine, threonine, tryptophan, tyrosine and valine, has a net charge of +2 to +8, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 12 to 25 amino acids selected from the group consisting of alanine, arginine, glutamic acid, glycine, isoleucine, leucine, lysine, phenylalanine, proline, serine, threonine, tryptophan, tyrosine and valine, has a net charge of +3 to +7, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 12 to 25 amino acids selected from the group consisting of alanine, arginine, glutamic acid, glycine, isoleucine, leucine, lysine, phenylalanine, proline, serine, threonine, tryptophan, tyrosine and valine, has a net charge of +4 to +6, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50, preferably 10 to 30, preferably 12 to 25, amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, preferably selected from alanine, arginine, glutamic acid, glycine, isoleucine, leucine, lysine, phenylalanine, proline, serine, threonine, tryptophan, tyrosine and valine, has a net charge of +2 to +8, preferably +3 to +7, preferably +4 to +6, and is antimicrobially active, the antimicrobial activity being determined as described in Example 1.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50 amino acids selected from the group consisting of alanine, arginine, glutamic acid, glycine, isoleucine, leucine, lysine, phenylalanine, proline, serine, threonine, tryptophan, tyrosine and valine, has a net charge of +2 to +8, and is antimicrobially active, the antimicrobial activity being determined as described in Example 1.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50 amino acids selected from the group consisting of alanine, arginine, glutamic acid, glycine, isoleucine, leucine, lysine, phenylalanine, proline, serine, threonine, tryptophan, tyrosine and valine, has a net charge of +3 to +7, and is antimicrobially active, the antimicrobial activity being determined as described in Example 1.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50 amino acids selected from the group consisting of alanine, arginine, glutamic acid, glycine, isoleucine, leucine, lysine, phenylalanine, proline, serine, threonine, tryptophan, tyrosine and valine, has a net charge of +4 to +6, and is antimicrobially active, the antimicrobial activity being determined as described in Example 1.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 30 amino acids selected from the group consisting of alanine, arginine, glutamic acid, glycine, isoleucine, leucine, lysine, phenylalanine, proline, serine, threonine, tryptophan, tyrosine and valine, has a net charge of +2 to +8, and is antimicrobially active, the antimicrobial activity being determined as described in Example 1.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 30 amino acids selected from the group consisting of alanine, arginine, glutamic acid, glycine, isoleucine, leucine, lysine, phenylalanine, proline, serine, threonine, tryptophan, tyrosine and valine, has a net charge of +3 to +7, and is antimicrobially active, the antimicrobial activity being determined as described in Example 1.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 30 amino acids selected from the group consisting of alanine, arginine, glutamic acid, glycine, isoleucine, leucine, lysine, phenylalanine, proline, serine, threonine, tryptophan, tyrosine and valine, has a net charge of +4 to +6, and is antimicrobially active, the antimicrobial activity being determined as described in Example 1.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 12 to 25 amino acids selected from the group consisting of alanine, arginine, glutamic acid, glycine, isoleucine, leucine, lysine, phenylalanine, proline, serine, threonine, tryptophan, tyrosine and valine, has a net charge of +2 to +8, and is antimicrobially active, the antimicrobial activity being determined as described in Example 1.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 12 to 25 amino acids selected from the group consisting of alanine, arginine, glutamic acid, glycine, isoleucine, leucine, lysine, phenylalanine, proline, serine, threonine, tryptophan, tyrosine and valine, has a net charge of +3 to +7, and is antimicrobially active, the antimicrobial activity being determined as described in Example 1.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 12 to 25 amino acids selected from the group consisting of alanine, arginine, glutamic acid, glycine, isoleucine, leucine, lysine, phenylalanine, proline, serine, threonine, tryptophan, tyrosine and valine, has a net charge of +4 to +6, and is antimicrobially active, the antimicrobial activity being determined as described in Example 1.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50, preferably 10 to 30, preferably 12 to 25, amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, preferably selected from alanine, arginine, glutamic acid, glycine, isoleucine, leucine, lysine, phenylalanine, proline, serine, threonine, tryptophan, tyrosine and valine, has a net charge of +2 to +8, preferably +3 to +7, preferably +4 to +6, and is antimicrobially active, the antimicrobial activity being determined as described in Example 1, wherein the peptide comprises at least 1 to 5 tryptophan functional groups and/or 1 to 5 arginine functional groups and/or 1 to 5 lysine functional groups, preferably at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50 amino acids selected from the group consisting of alanine, arginine, glutamic acid, glycine, isoleucine, leucine, lysine, phenylalanine, proline, serine, threonine, tryptophan, tyrosine and valine, has a net charge of +2 to +8, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50 amino acids selected from the group consisting of alanine, arginine, glutamic acid, glycine, isoleucine, leucine, lysine, phenylalanine, proline, serine, threonine, tryptophan, tyrosine and valine, has a net charge of +3 to +7, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 50 amino acids selected from the group consisting of alanine, arginine, glutamic acid, glycine, isoleucine, leucine, lysine, phenylalanine, proline, serine, threonine, tryptophan, tyrosine and valine, has a net charge of +4 to +6, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 30 amino acids selected from the group consisting of alanine, arginine, glutamic acid, glycine, isoleucine, leucine, lysine, phenylalanine, proline, serine, threonine, tryptophan, tyrosine and valine, has a net charge of +2 to +8, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 30 amino acids selected from the group consisting of alanine, arginine, glutamic acid, glycine, isoleucine, leucine, lysine, phenylalanine, proline, serine, threonine, tryptophan, tyrosine and valine, has a net charge of +3 to +7, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 10 to 30 amino acids selected from the group consisting of alanine, arginine, glutamic acid, glycine, isoleucine, leucine, lysine, phenylalanine, proline, serine, threonine, tryptophan, tyrosine and valine, has a net charge of +4 to +6, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 12 to 25 amino acids selected from the group consisting of alanine, arginine, glutamic acid, glycine, isoleucine, leucine, lysine, phenylalanine, proline, serine, threonine, tryptophan, tyrosine and valine, has a net charge of +2 to +8, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 12 to 25 amino acids selected from the group consisting of alanine, arginine, glutamic acid, glycine, isoleucine, leucine, lysine, phenylalanine, proline, serine, threonine, tryptophan, tyrosine and valine, has a net charge of +3 to +7, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In preferred embodiments, the peptide comprises an amino acid sequence that, over the entire length thereof, is at least 90% and increasingly preferably at least 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5, wherein the peptide has a length of 12 to 25 amino acids selected from the group consisting of alanine, arginine, glutamic acid, glycine, isoleucine, leucine, lysine, phenylalanine, proline, serine, threonine, tryptophan, tyrosine and valine, has a net charge of +4 to +6, and is antimicrobially active, the antimicrobial activity being determined as described in Example 1, wherein the peptide comprises at least 1 to 4 tryptophan functional groups and/or 1 to 3 arginine functional groups and/or 1 to 5 lysine functional groups.

In particularly preferred embodiments, the peptide comprises an amino acid sequence identical to one of the amino acid sequences set forth in SEQ ID NO:1, SEQ ID NO:2, SEQ ID NO:3, SEQ ID NO:4 or SEQ ID NO:5. In particularly preferred embodiments, the peptide comprises an amino acid sequence identical to one of the amino acid sequences set forth in SEQ ID NO:1, SEQ ID NO:2, SEQ ID NO:3, SEQ ID NO:4 or SEQ ID NO:5, wherein the peptide is antimicrobially active, the antimicrobial activity being determined as described in Example 1.

In particularly preferred embodiments, the peptide comprises an amino acid sequence identical to one of the amino acid sequences set forth in SEQ ID NO:1. In particularly preferred embodiments, the peptide comprises an amino acid sequence identical to one of the amino acid sequences set forth in SEQ ID NO:1, wherein the peptide is antimicrobially active, the antimicrobial activity being determined as described in Example 1.

In particularly preferred embodiments, the peptide comprises an amino acid sequence identical to one of the amino acid sequences set forth in SEQ ID NO:2. In particularly preferred embodiments, the peptide comprises an amino acid sequence identical to one of the amino acid sequences set forth in SEQ ID NO:2, wherein the peptide is antimicrobially active, the antimicrobial activity being determined as described in Example 1.

In particularly preferred embodiments, the peptide comprises an amino acid sequence identical to one of the amino acid sequences set forth in SEQ ID NO:3. In particularly preferred embodiments, the peptide comprises an amino acid sequence identical to one of the amino acid sequences set forth in SEQ ID NO:3, wherein the peptide is antimicrobially active, the antimicrobial activity being determined as described in Example 1.

In particularly preferred embodiments, the peptide comprises an amino acid sequence identical to one of the amino acid sequences set forth in SEQ ID NO:4. In particularly preferred embodiments, the peptide comprises an amino acid sequence identical to one of the amino acid sequences set forth in SEQ ID NO:4, wherein the peptide is antimicrobially active, the antimicrobial activity being determined as described in Example 1.

In particularly preferred embodiments, the peptide comprises an amino acid sequence identical to one of the amino acid sequences set forth in SEQ ID NO:5. In particularly preferred embodiments, the peptide comprises an amino acid sequence identical to one of the amino acid sequences set forth in SEQ ID NO:5, wherein the peptide is antimicrobially active, the antimicrobial activity being determined as described in Example 1.

Peptides and proteins are usually chains of amino acids, wherein the individual amino acids are linked to each other via peptide bonds. Chains of two amino acids are called dipeptides, chains of three amino acids are called tripeptides, chains of four amino acids are called tetrapeptides, etc. Chains of a few up to approximately 100 amino acids are called oligopeptides (up to approximately 10 amino acids) or polypeptides (approximately 10 to 100 amino acids) or simply peptides. Chains of more than approximately 100 amino acids are usually referred to as proteins.

Peptides according to the invention do not exhibit the catalytic activity typical of enzymes. The definition of peptides according to the invention therefore does not include enzymes. While enzymes sometimes have an antimicrobial effect because they can cleave 1,4-β-glycosidic compounds in cell wall and/or cell membrane components of microorganisms, the antimicrobial effect of the peptides described herein is not based on such an enzymatic-catalytic mode of action, but on, inter alia, non-enzymatic-catalytic damage to cell wall and/or cell membrane integrity and/or the blocking of vital metabolic pathways and/or the blocking of receptors. Antimicrobial peptides can vary greatly in their composition and structure and have been isolated from different organisms, e.g., vertebrates, invertebrates, insects, plants and microorganisms. Antimicrobial peptides initially interact electrostatically with the microbial cell wall or lipid membrane components and then often penetrate the membrane by forming voltage-dependent channels. In this way, “pores” form and can tear holes in the viral, bacterial or fungal envelope (see e.g., Bahar et al., Antimicrobial Peptides, Pharmaceuticals, 2013, 6, 1543-157).

Peptides according to the invention have a length of 10 to 50, preferably 10 to 30, preferably 12 to 25, amino acids. According to the invention, the amino acids are selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, preferably selected from alanine, arginine, glutamic acid, glycine, isoleucine, leucine, lysine, phenylalanine, proline, serine, threonine, tryptophan, tyrosine and valine.

Peptides according to the invention have a net charge of +2 to +8, preferably +3 to +7, preferably +4 to +6. The net charge of a peptide corresponds to the sum of the charge of the acidic (−1), basic (+1) and neutral amino acid side chain (0) at a pH value of 7.0.

Within the context of the invention, cleaning performance is understood to mean the ability of an agent to partially or completely remove an existing stain, in particular the lightening performance on one or more stains on textiles. Within the scope of the invention, both an agent and the washing/cleaning liquor formed by this agent have a respective cleaning performance. The cleaning performance is preferably ascertained as described hereafter.

The washing or cleaning liquor is understood to be the working solution containing the detergent or cleaning agent which acts on the textiles or fabrics or surfaces, in particular dishes or household surfaces, and thus comes into contact with the stains present on the textiles or fabrics or surfaces. The washing/cleaning liquor is usually formed when the washing/cleaning process begins and the agent is diluted with water, e.g., in a washing machine or dishwasher or in another suitable container.

The cleaning performance on textiles or fabrics can be determined in a washing system containing a detergent in a dosage between 2.0 and 8.0 grams per liter of washing liquor.

The concentration of the peptide according to the invention in the detergent intended for this washing system is 1×10⁻⁸ to 5 wt. %, preferably 0.0001 to 1 wt. %, more preferably 0.0005 to 0.5 wt. %, particularly preferably 0.001 to 0.1 wt. %, based on active protein and total weight of the detergent.

The protein concentration can be determined using known methods, e.g., the BCA method (bicinchoninic acid; 2,2′-bichinolyl-4,4′-dicarboxylic acid) or the Biuret method (Gornall et al. J. Biol. Chem., 1948, 177, 751-766). In this regard, the active protein concentration can be determined via titration of the active centers using a suitable irreversible inhibitor and determination of the residual activity (Bender et al., J. Am. Chem. Soc., 1966, 88, 24, 5890-5913).

A liquid reference detergent for such a washing system may be composed, for example, as follows (all figures in wt. %): 4.4% alkyl benzene sulfonic acid, 5.6% further anionic surfactants, 2.4% C₁₂₋₁₈ Na salts of fatty acids (soaps), 4.4% non-ionic surfactants, 0.2% phosphonates, 1.4% citric acid, 0.95% NaOH, 0.01% defoamer, 2% glycerol, 0.08% preservatives, 1% ethanol, and the remainder being demineralized water. The dosage of the liquid detergent is preferably between 3.0 and 6.0 grams per liter of washing liquor, e.g., 3.0, 3.2, 3.5, 3.7, 4.0, 4.5, 4.7, 4.9 or 5.9 grams per liter of washing liquor. The washing process preferably takes place in a pH range between pH 7 and pH 10.5, preferably between pH 8 and pH 9.

The cleaning performance against stains on textiles is determined by measuring the degree of cleaning of the washed textiles. For example, the washing process can take place for 60 minutes at a temperature of 40° C. and the water can have a water hardness between 15.5° dH and 16.5° dH (German hardness).

The degree of whiteness, i.e., the lightening of the stains, as a measure of the cleaning performance is determined using optical measuring methods, preferably photometrically. A suitable device for this purpose is, for example, the Minolta CM508d spectrometer. Usually, the devices used for measurement are calibrated beforehand using a white standard, preferably a supplied white standard.

The cleaning performance on surfaces, in particular household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets, can be determined as described in the German patent application 102023201692.3.

Preferred embodiments of uses and agents according to the invention achieve such advantageous cleaning performance even at low temperatures, preferably in a temperature range of about 10° C. to about 60° C., preferably about 15° C. to about 40° C., particularly preferably about 20° C. to about 30° C.

In preferred embodiments, peptides according to the invention can be used in detergents and cleaning agents as described herein. Such detergents and cleaning agents according to the invention include all conceivable types of detergents or cleaning agents, both concentrates and undiluted agents, for use on a commercial scale, in the washing machine or for hand washing or cleaning (hereinafter, the terms “detergent and/or “cleaning agent” and “agent” are used synonymously, unless explicit reference is made to a “detergent” or a “cleaning agent”). These include, for example, detergents for textiles, carpets or natural fibers, for which the term “detergent” is used. These include, for example, dishwashing detergents for dishwashers (automatic dishwasher detergents) or manual dishwashing detergents or cleaners for hard surfaces such as metal, glass, porcelain, ceramics, tiles, stone, painted surfaces, plastics, wood or leather, for which the term “cleaning agent” is used, i.e., in addition to manual and automatic dishwashing detergents, also, for example, scouring agents, glass cleaners, toilet fragrance rinser, etc. Detergents and cleaning agents within the scope of the invention also include washing additives which are added to the actual detergent during manual or automatic textile washing in order to achieve a further effect. Furthermore, within the scope of the invention, detergent and cleaning agents also include textile pre-treatment agents and post-treatment agents, i.e., agents with which the item of laundry is brought into contact before the actual washing, e.g., for dissolving stubborn soiling, and also agents which, in a step downstream of the actual textile washing, impart further desirable properties to the laundry item, such as a pleasant feel, crease resistance or low static charge. Inter alia, softeners are included in the latter agents. This also comprises agents for use in (semi-)automated washing or cleaning systems such as floor-mopping robots or wet vacuum cleaners.

Agents according to the invention, which may be in the form of powdery or granular solids, in compacted or re-compacted particulate form, homogeneous solutions or suspensions, may contain, in addition to the peptides according to the invention, all known ingredients that are conventional in such agents, wherein preferably at least one other ingredient is present in the agent. In particular, agents according to the invention can be surfactants, builders, complexing agents, polymers, glass corrosion inhibitors, corrosion inhibitors, bleaching agents such as peroxygen compounds, bleach activators or bleach catalysts, water-miscible organic solvents, enzymes, enzyme stabilizers, sequestering agents, electrolytes, pH regulators and/or other additives such as optical brighteners, greying inhibitors, dye transfer inhibitors, foam regulators as well as dyes and fragrances. Advantageous ingredients of agents according to the invention are disclosed in international patent application WO 2009/121725, starting at the penultimate paragraph of page 5 and ending after the second paragraph on page 13. Reference is expressly made to this disclosure and the disclosure therein is incorporated into the present patent application.

Agents according to the invention each advantageously contain the peptides according to the invention in an amount of from 2 g to 20 mg, preferably from 5 g to 17.5 mg, particularly preferably from 20 g to 15 mg and very particularly preferably from 50 g to 10 mg per g of the agent.

Agents according to the invention contain the peptides according to the invention increasingly preferably in an amount of from 1×10⁻⁸ to 5 wt. %, from 0.0001 to 1 wt. %, from 0.0005 to 0.5 wt. %, from 0.001 to 0.1 wt. %, based on active protein and total weight of the agent.

In preferred embodiments, agents according to the invention contain the peptides according to the invention in an amount of from 1×10⁻⁸ to 5 wt. %, preferably 0.0001 to 1 wt. %, preferably 0.0005 to 0.5 wt. %, particularly preferably 0.001 to 0.1 wt. %, based on active protein and total weight of the agent.

Further embodiments comprise all solid, powdery, liquid, gel-like or pasty dosage forms of agents according to the invention, which may optionally also consist of a plurality of phases and may be present in compressed or non-compressed form. The agent can be present as a free-flowing powder, in particular having a bulk density of 300 g/l to 1200 g/l, in particular 500 g/l to 900 g/l or 600 g/l to 850 g/l. The solid administration forms of the agent further include extrudates, granules, tablets or pouches. Alternatively, the agent can also be in a liquid, gel or pasty form, e.g., in the form of a non-aqueous liquid detergent or a non-aqueous paste or in the form of an aqueous liquid detergent or an aqueous paste. Liquid agents are generally preferred. Furthermore, the agent can be present as a single-component system. Such agents consist of one phase. Alternatively, an agent can also consist of a plurality of phases. Such an agent is accordingly divided into a plurality of components.

In a preferred embodiment, the agent according to the invention is a textile detergent.

In a preferred embodiment, the agent according to the invention is a liquid textile detergent.

In a preferred embodiment, the agent according to the invention is a pre-portioned detergent, in particular a detergent portion unit comprising a detergent preparation according to the invention and a water-soluble film which completely encloses the detergent preparation.

The water-soluble film in which the detergent preparation is packaged can comprise one or more structurally different water-soluble polymer(s). Suitable water-soluble polymer(s) are in particular polymers from the group of (optionally acetalized) polyvinyl alcohols (PVAL) and copolymers thereof. Water-soluble films are preferably based on a polyvinyl alcohol or a polyvinyl alcohol copolymer whose molecular weight is in the range of from 10,000 to 1,000,000 g/mol, preferably from 20,000 to 500,000 g/mol, particularly preferably from 30,000 to 100,000 g/mol, and in particular from 40,000 to 80,000 g/mol. Suitable water-soluble films for use are marketed, inter alia, by the company MonoSol LLC, for example, under the names M8630, M8720, M8310, C₈₄₀₀ or M8900. For example, films with the name Solublon® PT, Solublon® GA, Solublon® KC or Solublon® KL by Aicello Chemical Europe GmbH or the VF-HP films by Kuraray are also suitable.

If the agents according to the invention are in liquid form, they preferably contain more than 40 wt. %, preferably 50 to 90 wt. %, and particularly preferably 60 to 80 wt. %, of water, based on their total weight.

Agents according to the invention can contain one or more surfactants, with anionic surfactants, non-ionic surfactants and mixtures thereof being particularly suitable, although cationic, zwitterionic and/or amphoteric surfactants can also be contained. The agents preferably contain 5 to 70 wt. % surfactant, preferably 35 to 60 wt. %, and more preferably 40 to 55 wt. % surfactant, based on the total weight of the agent. In preferred embodiments, the agents preferably contain 3 to 35 wt. %, preferably 5 to 30 wt. %, of surfactant, based on the total weight of the agent.

Suitable anionic surfactants are, in particular, soaps and those which contain sulfate or sulfonate groups, preferably having alkali ions as cations. Usable soaps are preferably the alkali salts of saturated or unsaturated C₁₂₋₁₈ fatty acids. Fatty acids of this kind can also be used in a not completely neutralized form. Suitable sulfate-type surfactants include the salts of sulfuric acid half-esters of C₁₂₋₁₈ fatty alcohols atoms and the sulfation products of the mentioned non-ionic surfactants having a low degree of ethoxylation. Surfactants of the sulfonate type that can be used include, for example, C₉₋₁₄ alkylbenzene sulfonates, alkane sulfonates obtained from C₁₂₋₁₈ alkanes, for example by sulfochlorination or sulfoxidation with subsequent hydrolysis or neutralization, C₁₂₋₁₈ olefin sulfonates resulting from the reaction of corresponding monoolefins with sulfur trioxide, mixtures of alkene and hydroxyalkane sulfonates, disulfonates, such as those obtained from C₁₂₋₁₈ monoolefins with terminal or internal double bonds by sulfonation with gaseous sulfur trioxide and subsequent alkaline or acidic hydrolysis of the sulfonation products, and α-sulfofatty acid esters (ester sulfonates) resulting from the sulfonation of fatty acid methyl or ethyl esters, e.g., α-sulfonated methyl esters of hydrogenated coconut, palm kernel or tallow fatty acids.

Preferably, the agent comprises, based on the total weight of the agent, 2 to 55 wt. %, preferably 3 to 35 wt. %, of anionic surfactant. Very particularly preferably, the agent comprises 3 to 25 wt. % alkylbenzene sulfonate. In addition, the agent can preferably also contain other anionic surfactants, in particular alkyl ether sulfates, and non-ionic surfactants, in particular fatty alcohol alkoxylates. These can then make up the remainder of the surfactants.

Suitable alkylbenzene sulfonates are preferably selected from linear or branched alkylbenzene sulfonates of the formula

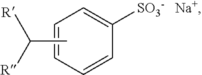

in which R′ and R″ are, independently of one another, hydrogen or alkyl, and together contain 6 to 19, preferably 7 to 15, and in particular 9 to 13, C atoms. A very particularly preferred representative is sodium dodecylbenzene sulfonate.

The alkali salts and in particular the sodium salts of the sulfuric acid half-esters of C₁₂₋₁₈ fatty alcohols, e.g. from coconut fatty alcohol, tallow fatty alcohol, lauryl alcohol, myristyl alcohol, cetyl alcohol or stearyl alcohol, or of C₁₀₋₂₀ oxo alcohols and the half-esters of secondary alcohols having these chain lengths are preferred as alk(en)yl sulfates. Alk(en)yl sulfates of the mentioned chain length that contain a synthetic straight-chain alkyl functional group prepared on a petrochemical basis and have a degradation behavior similar to that of the adequate compounds based on fat chemical raw materials are also preferred. From a washing perspective, the C₁₂₋₁₆ alkyl sulfates and C₁₂₋₁₅ alkyl sulfates and also C₁₄₋₁₅ alkyl sulfates are preferred.

The sulfuric acid monoesters of straight-chain or branched C₇₋₂₁ alcohols ethoxylated with 1 to 6 mol ethylene oxide, such as 2-methyl-branched C₉₋₁₁ alcohols with, on average, 3.5 mol ethylene oxide (EO) or C₁₂₋₁₈ fatty alcohols with 1 to 4 EO, are also suitable.

Suitable alkyl ether sulfates are, for example, compounds of the formula

R¹—O—(AO)ₙ—SO₃⁻X⁺.

In this formula, R¹ represents a linear or branched, substituted or unsubstituted alkyl functional group, preferably a linear, unsubstituted alkyl functional group, particularly preferably a fatty alcohol functional group. Preferred functional groups R¹ are selected from decyl, undecyl, dodecyl, tridecyl, tetradecyl, pentadecyl, hexadecyl, heptadecyl, octadecyl, nonadecyl, eicosyl functional groups and mixtures thereof, the representatives having an even number of C atoms being preferred. Particularly preferred functional groups R¹ are derived from C₁₂₋₁₈ fatty alcohols, for example from coconut fatty alcohol, tallow fatty alcohol, lauryl, myristyl, cetyl or stearyl alcohol or from C₁₀₋₂₀ oxo alcohols. AO represents an ethylene oxide (EO) group or propylene oxide (PO) group, preferably an ethylene oxide group. The index n represents an integer from 1 to 50, preferably from 1 to 20, and in particular from 2 to 10. Very particularly preferably, n represents the numbers 2, 3, 4, 5, 6, 7 or 8. X⁺ represents a monovalent cation or the n-th part of an n-valent cation, the alkali metal ions, including Na⁺ or K⁺, being preferred in this case, with Na⁺ being most preferred. Further cations X⁺ can be selected from NH₄⁺, ½Zn²⁺, ½Mg²⁺, ½Ca²⁺, ½Mn²⁺ and the mixtures thereof.

In various embodiments, the alkyl ether sulfate can be selected from fatty alcohol ether sulfates of the formula

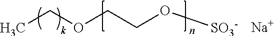

where k=11 to 19, and n=2, 3, 4, 5, 6, 7 or 8. Very particularly preferred representatives are Na—C₁₂₋₁₄ fatty alcohol ether sulfates having 2 EO (k=11-13, n=2). The degree of ethoxylation indicated represents a statistical average value which can be an integer or a fractional number for a specific product. The degrees of alkoxylation indicated represent statistical averages which can be an integer or a fractional number for a specific product. Preferred alkoxylates/ethoxylates have a narrowed homolog distribution (narrow range ethoxylates, NRE).

It has proven advantageous for cold wash performance if the agents additionally contain soap(s). Preferred agents are therefore characterized in that they contain soap(s). Saturated fatty acid soaps are suitable, such as the salts of lauric acid, myristic acid, palmitic acid, stearic acid, hydrogenated erucic acid and behenic acid, and in particular soap mixtures derived from natural fatty acids, such as coconut fatty acids, palm kernel fatty acids or tallow fatty acids.

Suitable non-ionic surfactants are in particular alkyl glycosides and ethoxylation and/or propoxylation products of alkyl glycosides or linear or branched alcohols each having 8 to approximately 18 C atoms in the alkyl portion and 3 to 20, preferably 4 to 10, alkyl ether groups. Furthermore, corresponding ethoxylation and/or propoxylation products of N-alkylamines, vicinal diols, fatty acid esters, and fatty acid amides, which correspond to the above-mentioned long-chain alcohol derivatives with respect to the alkyl moiety, and of alkylphenols having 5 to 12 C atoms in the alkyl radical, may be used.

Non-ionic surfactants that are preferably used are alkoxylated, advantageously ethoxylated, in particular primary alcohols having preferably 8 to 18 C atoms and, on average, 1 to 12 mol ethylene oxide (EO) per mol of alcohol, in which the alcohol functional group can be linear or preferably methyl-branched in the 2-position, or can contain linear and methyl-branched functional groups in the mixture, as are usually present in oxo alcohol functional groups. However, alcohol ethoxylates having linear functional groups of alcohols of native origin having 12 to 18 C atoms, for example of coconut, palm, tallow fatty or oleyl alcohol, and an average of 2 to 8 EO per mol of alcohol, are particularly preferred. Preferred ethoxylated alcohols include, for example, C₁₂₋₁₄ alcohols having 3 EO or 4 EO, C₉₋₁₁ alcohols having 7 EO, C₁₃₋₁₅ alcohols having 3 EO, 5 EO, 7 EO or 8 EO, C₁₂₋₁₈ alcohols having 3 EO, 5 EO or 7 EO, and mixtures thereof, such as mixtures of C₁₂₋₁₄ alcohol having 3 EO and C₁₂₋₁₈ alcohol having 5 EO. The degrees of ethoxylation specified represent statistical averages that can correspond to an integer or a fractional number for a specific product. Preferred alcohol ethoxylates have a narrowed homolog distribution (narrow range ethoxylates, NRE). In addition to these non-ionic surfactants, fatty alcohols having more than 12 EO can also be used. Examples of these are tallow fatty alcohols having 14 EO, 25 EO, 30 EO, or 40 EO.

Another class of non-ionic surfactants that are preferably used, which are used either as the sole non-ionic surfactant or in combination with other non-ionic surfactants, is alkoxylated, preferably ethoxylated or ethoxylated and propoxylated fatty acid alkyl esters, preferably having 1 to 4 carbon atoms in the alkyl chain, in particular fatty acid methyl esters.

Another class of non-ionic surfactants that can advantageously be used is the alkyl polyglycosides (APG). Usable alkyl polyglycosides satisfy the general formula

RO(G)_{z},

in which R is a linear or branched, in particular methyl-branched in the 2-position, saturated or unsaturated, aliphatic functional group having 8 to 22, preferably 12 to 18 C atoms and G is the symbol which represents a glycose unit having 5 or 6 C atoms, preferably glucose. The degree of glycosidation z is between 1 and 4, preferably between 1 and 2, and in particular between 1.1 and 1.4. Linear alkyl polyglycosides, i.e., alkyl polyglycosides in which the polyglycol functional group is a glucose functional group and the alkyl functional group is an n-alkyl functional group, are preferably used.

Non-ionic surfactants of the aminoxide type, for example N-cocoalkyl-N,N-dimethylamine oxide and N-tallow-alkyl-N,N-dihydroxyethylamine oxide, and of the fatty acid alkanolamides may also be suitable. The quantity of these non-ionic surfactants is preferably no more than that of the ethoxylated fatty alcohols, in particular no more than half thereof.

Suitable amphoteric surfactants are, for example, betaines of the formula

(Rⁱⁱⁱ)(R^{iv})(R^{v})N⁺CH₂COO⁻,

in which Rⁱⁱⁱ is an alkyl functional group having 8 to 25, preferably 10 to 21 carbon atoms, which may be interrupted by heteroatoms or heteroatom groups, and R^{iv} and R^{v} are identical or different alkyl functional groups having 1 to 3 carbon atoms, in particular C₁₀₋₁₈ alkyl dimethyl carboxymethyl betaine and C₁₋₁₇ alkylamidopropyldimethyl carboxymethyl betaine.

Suitable cationic surfactants include, inter alia, the quaternary ammonium compounds of the formula

(R^{vi})(R^{vii})(R^{viii})(R^{ix})N⁺X⁻,

in which R^{vi} to R^{ix} represent four identical or different, in particular two long-chain and two short-chain, alkyl functional groups and X⁻ represents an anion, in particular a halide ion, e.g., didecyldimethylammonium chloride, alkylbenzyldidecylammonium chloride and mixtures thereof. Further suitable cationic surfactants are the quaternary surface-active compounds, in particular having a sulfonium, phosphonium, iodonium or arsonium group, which are also known as antimicrobial active ingredients. By using quaternary surface-active compounds having an antimicrobial effect, the agent can be provided with an antimicrobial effect or the antimicrobial effect that may already be present due to other ingredients can be improved.

In preferred embodiments, the detergent and cleaning agent according to the invention comprises, in each case based on the total weight of the agent,
(i) 2 to 20 wt. %, preferably 3 to 17 wt. %, of anionic surfactants,
(ii) 1 to 10 wt. %, preferably 3 to 8 wt. %, of non-ionic surfactants,
(iii) 0 to 1 wt. %, preferably 0 to 0.5 wt. %, of soap, and
(iv) 0 to 5 wt. %, preferably 0 to 3 wt. %, of fatty acids.

Complexing agents are another preferred component of agents according to the invention. Particularly preferred complexing agents are the phosphonates, provided that their use is permitted by regulations. In addition to 1-hydroxyethane-1,1-diphosphonic acid, the complexing phosphonates include a number of different compounds such as diethylenetriamine penta(methylene phosphonic acid) (DTPMP). Hydroxyalkane or aminoalkane phosphonates are particularly preferred in this application. Among the hydroxyalkane phosphonates, 1-hydroxyethane-1,1-diphosphonate (HEDP) is of particular importance as a cobuilder. It is preferably used as a sodium salt, the disodium salt reacting neutrally and the tetrasodium salt reacting alkaline (pH 9). Possible amino alkane phosphonates preferably include ethylenediamine tetramethylene phosphonate (EDTMP), diethylenetriamine pentamethylene phosphonate (DTPMP) and the higher homologs thereof. They are preferably used in the form of the neutrally reacting sodium salts, e.g., as hexasodium salt of EDTMP or as the heptasodium and octasodium salt of DTPMP. Of the class of phosphonates, HEDP is preferably used as a builder. The aminoalkane phosphonates also have a pronounced heavy metal binding capacity. Accordingly, it may be preferred, in particular if the agents also contain bleach, to use aminoalkane phosphonates, in particular DTPMP, or to use mixtures of the aforementioned phosphonates. A preferred agent in the context of this application contains one or more phosphonate(s) from the group aminotrimethylene phosphonic acid (ATMP) and/or the salts thereof; ethylenediamine tetra(methylene phosphonic acid) (EDTMP) and/or the salts thereof; diethylenetriamine penta(methylene phosphonic acid) (DTPMP) and/or the salts thereof; 1-hydroxyethane-1,1-diphosphonic acid (HEDP) and/or the salts thereof; 2-phosphonobutane-1,2,4-tricarboxylic acid (PBTC) and/or the salts thereof; hexamethylenediamine tetra(methylene phosphonic acid) (HDTMP) and/or the salts thereof; nitrilotri(methylenephosphonic acid) (NTMP) and/or the salts thereof. Agents containing 1-hydroxyethane-1,1-diphosphonic acid (HEDP) or diethylenetriaminepenta(methylenephosphonic acid) (DTPMP) as phosphonates are particularly preferred. According to the invention agents may, of course, contain two or more different phosphonates.

Agents that are preferred according to the invention are characterized in that the agent contains at least one complexing agent from the group of phosphonates, preferably 1-hydroxyethane-1,1-diphosphonate, the proportion by weight of the phosphonate with respect to the total weight of the agent preferably being between 0.1 and 8.0 wt. %, more preferably 0.2 and 5.0 wt. %, even more preferably 0.3 and 3.0 wt. %, and particularly preferably 0.5-2.0 wt. %.

In further preferred embodiments, agents according to the invention are substantially free of phosphonate-containing compounds. “Substantially free of phosphonate-containing compounds” in this context means that the corresponding agents or compositions contain less than 2 wt. %, preferably less than 1 wt. %, more preferably less than 0.5 wt. %, and particularly preferably less than 0.1 wt. %, of phosphonate-containing compounds, based on the total weight of the agent. In particularly preferred embodiments, these agents/compositions are free from phosphonate-containing compounds.

Agents according to the invention further preferably contain builders, preferably at least one water-soluble and/or water-insoluble, organic and/or inorganic builder. The builders include in particular the silicates, carbonates and/organic cobuilders.

Polycarboxylates/polycarboxylic acids, polymeric polycarboxylates, aspartic acid, polyacetals, dextrins, other organic cobuilders and phosphonates are particularly noteworthy as organic cobuilders. These substance classes are described below. Organic cobuilder substances can, if desired, be contained in amounts of up to 40 wt. %, in particular up to 25 wt. %, and preferably from 1 to 8 wt. %, based on the total weight of the agent. Suitable organic builder substances are, for example, the polycarboxylic acids that can be used in the form of the free acids and/or the sodium salts thereof, where polycarboxylic acids are understood to mean the carboxylic acids which carry more than one acid function. For example, these are citric acid, adipic acid, succinic acid, glutaric acid, malic acid, tartaric acid, maleic acid, fumaric acid, saccharic acid and carboxymethyl inulines, monomeric and polymeric aminopolycarboxylic acids, in particular glycine diacetic acid, methylglycine diacetic acid, nitrilotriacetic acid (NTA), iminodisuccinates such as ethylenediamine-N,N′-disuccinic acid and hydroxyiminodisuccinates, ethylenediaminetetraacetic acid and polyaspartic acid, polyphosphonic acids, in particular aminotris(methylenephosphonic acid), ethylenediamine tetrakis(methylenephosphonic acid), lysine tetra(methylenephosphonic acid) and 1-hydroxyethane-1,1-diphosphonic acid, polymeric hydroxy compounds such as dextrin, and polymeric (poly)carboxylic acids, polycarboxylates which can be obtained in particular by oxidizing polysaccharides or dextrins, and/or polymeric acrylic acids, methacrylic acids, maleic acids, and mixed polymers thereof, which may also contain small amounts of polymerizable substances without carboxylic acid functionality in polymerized form. If desired, such organic builder substances can be contained in amounts of up to 50 wt. %, in particular up to 25 wt. %, preferably from 10 to 20 wt. %, and particularly preferably from 1 to 5 wt. %, based on the total weight of the agent. In addition to their builder effect, the free acids typically also have the property of being an acidification component and are thus also used for setting a lower and milder pH of agents. Particularly noteworthy here are citric acid, succinic acid, glutaric acid, adipic acid, gluconic acid, and any mixtures thereof. Citric acid or salts of citric acid are particularly preferably used as builder substances. Further particularly preferred builder substances are selected from methylglycinediacetic acid (MGDA), glutamic acid diacetate (GLDA), aspartic acid diacetate (ASDA), hydroxyethyl iminodiacetate (HEIDA), iminodisuccinate (IDS), ethylenediamine disuccinate (EDDS), carboxymethyl inulin and polyaspartate.

In preferred embodiments, citric acid and/or citrate is used as the water-soluble, organic builder. Particularly preferred is the use, based on the total weight of the agent, of 0.5 to 25 wt. %, preferably 0.75 to 12.5 wt. %, more preferably 1 to 4 wt. % of citric acid and/or 0.5 to 25 wt. %, preferably 0.75 to 12.5 wt. %, more preferably 1 to 4 wt. % of citrate, preferably alkali citrate, even more preferably sodium citrate. Citric acid/citrate can each be used in the form of their hydrates, for example citric acid can be used in the form of the monohydrate, and citrate can be used in the form of the trisodium citrate dihydrate.

In more preferred embodiments, the builder substances are selected from MGDA and GLDA. As used herein, the term “MGDA” comprises, but is not limited to, methylglycine diacetic acid, α-alanine diacetic acid, N-(1-carboxyethyl)-iminodiacetic acid and N,N-bis(carboxymethyl)-DL-alanine, wherein the free acid forms and the corresponding salts, preferably alkali salts, in particular trisodium salts, are included. As used herein, the term “GLDA” includes, but is not limited to, glutamic acid diacetic acid, L-glutamic acid-N,N-diacetic acid and N,N-bis(carboxylatomethyl)-L-glutamate, wherein free acid forms and corresponding salts, preferably alkali salts, in particular tetrasodium salts, are included. Although, based on the total weight of the agent, higher MGDA or GLDA concentrations are possible, in particular up to 25 wt. %, 0.2 to 5 wt. %, preferably 0.25 to 3 wt. %, even more preferably 0.5 to 2 wt. % MGDA, preferably MGDA trisodium salt (MGDA-Na₃) is used. Even more preferable is the use, based on the total weight of the agent, of 0.2 to 5 wt. %, preferably 0.25 to 3 wt. %, even more preferably 0.5 to 2 wt. % of GLDA, preferably GLDA tetrasodium salt (GLDA-Na₄).

Polymeric polycarboxylates are also suitable as builders, e.g., the alkali metal salts of polyacrylic acid or polymethacrylic acid, e.g., those with a relative molecular mass of 500 to 70,000 g/mol. For the purpose of this application, the molar masses indicated for polymeric polycarboxylates are weight-average molar masses Mw of the respective acid form which have been determined in principle using gel permeation chromatography (GPC), a UV detector having been used. The measurement was carried out against an external polyacrylic acid standard which, due to the structural relationship to the tested polymers, yields realistic molecular weight values. These specifications differ significantly from the molecular weight specifications for which polystyrene sulfonic acids are used as the standard. The molar masses measured against polystyrene sulfonic acids are generally considerably higher than the molar masses indicated in this application. Suitable polymers are in particular polyacrylates which preferably have a molecular mass of from 2,000 to 20,000 g/mol. Due to their superior solubility, short-chain polyacrylates having molar masses of from 2,000 to 10,000 g/mol, and particularly preferably of from 3,000 to 5,000 g/mol, may in turn be preferred from this group. In addition, copolymeric polycarboxylates are suitable, in particular those of acrylic acid with methacrylic acid and those of acrylic acid or methacrylic acid with maleic acid. Copolymers of acrylic acid with maleic acid containing 50 to 90 wt. % acrylic acid and 50 to 10 wt. % maleic acid have proven to be particularly suitable. The relative molecular mass thereof, based on free acids, is generally 2,000 to 70,000 g/mol, preferably 20,000 to 50,000 g/mol, and in particular 30,000 to 40,000 g/mol.

A solid agent according to the invention preferably contains at least one water-soluble and/or water-insoluble, organic and/or inorganic builder. The water-soluble organic builder substances include the aforementioned organic builder substances.

In addition to the water-soluble organic builders mentioned above, the agents of the invention may also further contain inorganic water-soluble builders. In particular, alkali silicates, alkali carbonates, alkali hydrogen carbonates, alkali phosphates and/or sesquicarbonates, which can be present in the form of their alkaline, neutral, or acidic sodium or potassium salts, can be used as water-soluble inorganic builder materials. Small amounts of calcium carbonate may optionally also be contained in solid textile detergents. Water-soluble crystalline and/or amorphous alkali silicates are suitable, for example. The alkali silicates that can be used in the agents according to the invention as builders preferably have a molar ratio of alkali oxide to SiO₂ of less than 0.95, in particular of from 1:1.1 to 1:12, and may be present in amorphous or crystalline form. Preferred alkali silicates are sodium silicates, in particular amorphous sodium silicates, having a molar ratio of Na₂O:SiO₂ of from 1:2 to 1:2.8. As crystalline silicates, which can be present alone or in a mixture with amorphous silicates, crystalline phyllosilicates of the general formula Na₂SiₓO₂ₓ₊₁·y H₂O are preferably used, in which x, known as the modulus, is a number from 1.9 to 22, in particular 1.9 to 4, and y is a number from 0 to 33, and preferred values for x are 2, 3 or 4. Preferred crystalline phyllosilicates are those in which x assumes the values 2 or 3 in the mentioned general formula. In particular, both β- and δ-sodium disilicates (Na₂Si₂O₅·y H₂O) are preferred. Practically water-free crystalline alkali silicates of the above general formula, in which x is a number from 1.9 to 2.1 and which are produced from amorphous alkali silicates, may also be used in agents according to the invention. In a further embodiment of agents according to the invention, a crystalline sodium phyllosilicate having a module of from 2 to 3, as can be prepared from sand and soda, is used. Crystalline sodium silicates having a module in the range of from 1.9 to 3.5 are used in a further embodiment of agents according to the invention. In agents containing amorphous and crystalline alkali silicates, the weight ratio of amorphous alkali silicate to crystalline alkali silicate is preferably from 1:2 to 2:1 and in particular from 1:1 to 2:1. Crystalline phyllosilicates of the above formula are sold by Clariant GmbH under the trade name Na-SKS, for example Na-SKS-1 (Na₂Si₂₂O₄₅·x H₂O, kenyaite), Na-SKS-2 (Na₂Si₁₄O₂₉·x H₂O, magadiite), Na-SKS-3 (Na₂S₁₈O₁₇·x H₂O) or Na-SKS-4 (Na₂Si₄O₉·x H₂O, macatite). Of these, Na-SKS-5 (α-Na₂Si₂O₅), Na-SKS-7 (β-Na₂Si₂O₅, natrosilite), Na-SKS-9 (NaHSi₂O₅·3 H₂O), Na-SKS-10 (NaHSi₂O₅·3 H₂O, kanemite), Na-SKS-11 (t-Na₂Si₂O₅) and Na-SKS-13 (NaHSi₂O₅), but in particular Na-SKS-6 (δ-Na₂Si₂O₅) are particularly suitable. In one embodiment of agents according to the invention, a granular compound made of crystalline phyllosilicate and citrate, crystalline phyllosilicate and the above-described (co)polymeric polycarboxylic acid, or alkali silicate and alkali carbonate is used, as is commercially available under the name Nabion® 15, for example. Such water-soluble inorganic builder materials are preferably contained in the agents according to the invention in amounts of 1 to 20 wt. %, in particular 5 to 15 wt. %, based on the total weight of the agent. Also of significance as water-soluble inorganic builder substances are the carbonates (and hydrogen carbonates), in particular sodium carbonate, and the phosphonic acids/phosphonates.

Agents according to the invention are preferably free of phosphate builders, i.e., contain, based on the total weight of the agent, less than 1 wt. % preferably no intentionally added phosphate builder.

Agents according to the invention may also contain water-insoluble builder substances. Crystalline or amorphous water-dispersible alkali aluminosilicates, in amounts of up to 50 wt. %, preferably not more than 40 wt. %, in particular from 3 to 20 wt. %, and particularly preferably from 1 to 15 wt. %, based on the total weight of the agent, are used in particular as water-insoluble inorganic builder materials. Among these, the crystalline sodium aluminosilicates in detergent quality, in particular zeolite A, zeolite P, zeolite MAP and optionally zeolite X, either alone or in mixtures, for example in the form of a co-crystallizate of the zeolites A and X (Vegobond® AX, a commercial product from Condea Augusta S.p.A.), are preferred. Amounts close to the stated upper limit are preferably used in solid, particulate agents. Suitable aluminosilicates have, in particular, no particles having a particle size above 30 μm and preferably consist by at least 80 wt. % of particles having a size below 10 μm. The calcium binding capacity, which can be determined according to DE 2412837 A1, of said aluminosilicates is generally in the range of from 100 to 200 mg CaO per gram.

In preferred embodiments, agents according to the invention comprise a builder system comprising at least one builder, preferably in an amount of 0.5 to 50 wt. %, preferably 0.5 to 20 wt. %, particularly preferably 0.5 to 10 wt. %, based on the total weight of the agent, wherein the builder is selected from the group consisting of polycarboxylic acids such as citric acid, adipic acid, succinic acid, glutaric acid, malic acid, tartaric acid, maleic acid, fumaric acid, saccharic acids and carboxymethyl inulines or salts thereof, monomeric and polymeric aminopolycarboxylic acids such as glycine diacetic acid, methylglycine diacetic acid (MGDA), glutamic acid diacetic acid (GLDA), nitrile triacetic acid, iminodisuccinate such as ethylenediamine-N,N′-disuccinic acid and hydroxyiminodisuccinates, ethylenediaminetetraacetic acid and polyaspartic acid or salts thereof, polyphosphonic acids such as aminotris(methylenephosphonic acid), ethylenediaminetetrakis(methylenephosphonic acid), lysinetetra(methylenephosphonic acid), diethylenetriaminepenta(methylenephosphonic acid) (DTPMP) and 1-hydroxyethane-1,1-diphosphonic acid (HEDP) or salts thereof, polymeric hydroxy compounds such as dextrin, and mixtures thereof.

In preferred embodiments, the agent according to the invention comprises, in each case based on the total weight of the agent,
(i) 0 to 10 wt. %, preferably 1 to 4 wt. %, of citric acid and/or citrate, preferably alkali citrate,
(ii) 0 to 40 wt. %, preferably 0 to 15 wt. %, more preferably 1 to 3 wt. %, of alkali carbonate, preferably sodium carbonate,
(iii) 0 to 20 wt. %, preferably 3 to 10 wt. %, of alkali silicate,
(iv) 0 to 10 wt. %, preferably 0.5 to 2 wt. %, of phosphonic acid and/or alkali phosphonate, particularly preferably HEDP and/or DTPMP, and/or
(v) 0 to 10 wt. %, preferably 0.5 to 3 wt. %, of aminopolycarboxylic acids, preferably MGDA and/or GLDA.

In addition to the builders described above, the agent may contain cleaning-active polymers. The proportion by weight of the cleaning-active polymers with respect to the total weight of agents according to the invention is preferably 0.1 to 20 wt. %, more preferably 1.0 to 15 wt. %, and even more preferably 2.0 to 12 wt. %.

Possible peroxygen compounds suitable for use in the agents according to the invention include, in particular, organic peroxy acids or peracid salts of organic acids, such as phthalimidopercaproic acid, perbenzoic acid, or salts of diperdodecanedioic acid, hydrogen peroxide and inorganic salts releasing hydrogen peroxide under the washing conditions, which salts include perborate, percarbonate, persilicate, and/or persulfates such as caroate, as well as hydrogen peroxide inclusion compounds such as H₂O₂-urea adducts. Hydrogen peroxide can also be produced by means of an enzymatic system, i.e., an oxidase and the substrate thereof. If solid peroxygen compounds are intended to be used, these may be used in the form of powders or granules, which may also be coated in a manner known in principle. The peroxygen compounds can be added to the washing liquor as such or in the form of the agents containing them, which in principle can contain all conventional washing, cleaning or disinfectant components. Particularly preferably, alkali percarbonate or alkali perborate monohydrate is used. If an agent according to the invention contains peroxygen compounds, they are present in amounts of preferably up to 50 wt. %, in particular from 5 to 30 wt. %, more preferably from 0.1 to 20 wt. %, based on the total weight of the agent.

In preferred embodiments, agents according to the invention are substantially free of peroxygen compounds. “Substantially free from peroxygen-containing compounds” in this context means that the corresponding agents or compositions contain less than 2 wt. %, preferably less than 1 wt. %, more preferably less than 0.5 wt. %, and particularly preferably less than 0.1 wt. %, of peroxygen-containing compounds, based on the total weight of the agent/composition. In particularly preferred embodiments, these agents/compositions are free from peroxygen-containing compounds.

Compounds which, under perhydrolysis conditions, result in aliphatic peroxocarboxylic acids having preferably 1 to 10 C atoms, in particular 2 to 4 C atoms, and/or optionally substituted perbenzoic acid, may be used in the agents as bleach activators. Suitable substances are those which carry O- and/or N-acyl groups of the stated number of C atoms and/or optionally substituted benzoyl groups. Preferred are polyacylated alkylene diamines, in particular tetraacetylethylenediamine (TAED), acylated triazine derivatives, in particular 1,5-diacetyl-2,4-dioxohexahydro-1,3,5-triazine (DADHT), acylated glycolurils, in particular tetraacetylglycoluril (TAGU), N-acylimides, in particular N-nonanoylsuccinimide (NOSI), acylated phenol sulfonates or carboxylates or the sulfonic or carboxylic acids thereof, in particular nonanoyloxybenzenesulfonate or isononanoyloxybenzenesulfonate or laroyloxybenzenesulfonate (NOBS or iso-NOBS or LOBS), 4-(2-decanoyloxyethoxycarbonyloxy)-benzenesulfonate (DECOBS) or decanoyloxybenzoate (DOBA), carboxylic acid anhydrides, in particular phthalic acid anhydride, acylated polyhydric alcohols, in particular triacetin, ethylene glycol diacetate, 2,5-diacetoxy-2,5-dihydrofuran and enol esters, as well as acetylated sorbitol and mannitol or the described mixtures thereof (SORMAN), acylated sugar derivatives, in particular pentaacetyl glucose (PAG), pentaacetyl fructose, tetraacetylxylose and octaacetyl lactose, acetylated, optionally N-alkylated glucamine and gluconolactone, N-acylated lactams, for example N-benzoylcaprolactam, nitriles from which perimidic acids are formed, in particular aminoacetonitrile derivatives having a quaternized nitrogen atom, and/or oxygen-transferring sulfonimines and/or acylhydrazones. The hydrophilically substituted acyl acetals and the acyl lactams are likewise preferably used. Combinations of conventional bleach activators can also be used. Such bleach activators can, in particular in the presence of the aforementioned hydrogen peroxide-yielding bleaching agents, be present in the customary quantity range, preferably in amounts of from 0.5 to 10 wt. %, and in particular 1 to 8 wt. %, based on the total weight of the agent, but are preferably entirely absent when percarboxylic acid is used as the sole bleaching agent.

In addition to or instead of the conventional bleach activators, sulfonimines and/or bleach-boosting transition metal salts or transition metal complexes may also be contained in solid agents as what are referred to as bleach catalysts.

In preferred embodiments, agents according to the invention are substantially free of bleach activators and/or are free of bleach catalysts. “Substantially free” in this context means that the corresponding agents or compositions contain, based on the total weight of the agent, less than 2 wt. %, preferably less than 1 wt. %, more preferably less than 0.5 wt. %, and particularly preferably less than 0.1 wt. %, of bleach activators and/or bleach catalysts. In particularly preferred embodiments, these agents/compositions are free of bleach activators and/or bleach catalysts.

In preferred embodiments, agents according to the invention are substantially free of bleach, i.e., substantially free of any bleaching substances, in particular free of the peroxygen compounds, bleach activators and bleach catalysts described above. “Substantially free” in this context means that the corresponding agents or compositions contain, based on the total weight of the agent, less than 2 wt. %, preferably less than 1 wt. %, more preferably less than 0.5 wt. %, and particularly preferably less than 0.1 wt. %, of bleach, i.e., any bleaching substances, in particular the peroxygen compounds, bleach activators and bleach catalysts described above. In particularly preferred embodiments, these agents/compositions are free of bleach, i.e., free of any bleaching substances, in particular free of the peroxygen compounds, bleach activators and bleach catalysts described above.

Suitable graying inhibitors or soil release active ingredients (soil release polymer) are cellulose ethers, such as carboxymethyl cellulose, methyl cellulose, hydroxyalkyl celluloses and mixed cellulose ethers, such as methyl hydroxyethyl cellulose, methylhydroxypropyl cellulose and methyl carboxymethyl cellulose. Preferably, sodium carboxymethyl cellulose, hydroxypropyl methyl cellulose and mixtures thereof and, optionally, mixtures thereof with methyl cellulose are used. The soil release active ingredients commonly used include copolyesters containing dicarboxylic acid units, alkylene glycol units and polyalkylene glycol units. The proportion of graying inhibitors and/or soil release active ingredients in agents according to the invention is generally no greater than 2 wt. % and is preferably 0.5 to 1.5 wt. %, particularly preferably 0.5 to 2 wt. %, based on the total weight of the agent.

Derivatives of diaminostilbene disulfonic acid or the alkali metal salts thereof can be contained, for example, as optical brighteners, in particular for textiles made of cellulose fibers (e.g., cotton). Salts of 4,4′-bis(2-anilino-4-morpholino-1,3,5-triazinyl-6-amino)stilbene-2,2′-disulfonic acid or compounds having a similar structure which, instead of the morpholino group, have a diethanolamino group, a methylamino group, or a 2-methoxyethylamino group are suitable, for example. Furthermore, brighteners of the substituted 4,4′-distyryl-diphenyl type can be present, e.g., 4,4′-bis-(4-chloro-3-sulfostyryl)-diphenyl. Mixtures of brighteners can also be used. Brighteners of the 1,3-diaryl-2-pyrazoline type, e.g., 1-(p-sulfoamoylphenyl)-3-(p-chlorophenyl)-2-pyrazoline, and compounds having a similarly structure are particularly suitable for polyamide fibers. The content of the agent of optical brighteners or brightener mixtures in the agent is generally no greater than 1 wt. %, preferably 0.05 to 0.5 wt. %, based on the total weight of the agent. In a preferred embodiment of the invention, the agent is free of such active ingredients.

The customary foam regulators that can be used in the agents according to the invention include, for example, polysiloxane-silicic acid mixtures, the finely divided silicic acid contained therein preferably being silanized or otherwise hydrophobized. The polysiloxanes can consist of both linear compounds and crosslinked polysiloxane resins and mixtures thereof. Further defoamers are paraffinic hydrocarbons, in particular microparaffins and paraffin waxes of which the melting point is above 40° C., saturated fatty acids or soaps having in particular 20 to 22 C atoms, for example sodium behenate, and alkali salts of phosphoric acid mono- and/or dialkyl esters, in which the alkyl chains each have 12 to 22 C atoms. Among these, sodium monoalkyl phosphate and/or dialkyl phosphate having C₁₆₋₁₈ alkyl groups is preferably used. The proportion of foam regulators can preferably be 0.2 to 2 wt. %, particularly preferably not greater than 1 wt. %, based on the total weight of the agent.

In order to set the desired pH, agents according to the invention can contain acids that are compatible with the system and environmentally friendly, in particular citric acid, acetic acid, tartaric acid, malic acid, lactic acid, glycolic acid, succinic acid, glutaric acid, and/or adipic acid, but also mineral acids, in particular sulfuric acid or alkali hydrogen sulfates, or bases, in particular ammonium or alkali hydroxides, preferably sodium hydroxide. These types of pH regulators are contained in the agents according to the invention in amounts preferably no greater than 10 wt. %, in particular from 0.5 to 6 wt. %, particularly preferably from 0.3 to 2 wt. %, based on the total weight of the agent.

As a further component, agents according to the invention may contain an organic solvent. Adding organic solvents has an advantageous effect on the enzyme stability and cleaning performance of these agents. Preferred organic solvents are derived from the group of monohydric or polyhydric alcohols, alkanolamines or glycol ethers. The solvents are preferably selected from ethanol, n- or i-propanol, butanol, glycol, propanediol, butanediol, glycerol, diglycol, propylene diglycol, butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether, propylene glycol propyl ether, dipropylene glycol methyl ether, dipropylene glycol ethyl ether, methoxytriglycol, ethoxytriglycol, butoxytriglycol, 1-butoxyethoxy-2-propanol, 3-methyl-3-methoxybutanol, propylene-glycol-t-butyl ether and mixtures of these solvents. The proportion by weight of these organic solvents with respect to the total weight of the agents according to the invention is preferably 0.1 to 10 wt. %, more preferably 0.2 to 8.0 wt. %, and even more preferably 0.5 to 5.0 wt. %. A particularly preferred organic solvent which is particularly effective in stabilizing the cleaning agents is glycerol, as well as 1,2-propylene glycol. Liquid agents preferably comprise at least one polyol, preferably from the group glycerol and 1,2-propylene glycol, based on the total weight of the agent, preferably in an amount of from 0.1 to 10 wt. %, preferably 0.2 to 8.0 wt. %, and more preferably 0.5 to 5.0 wt. %. Other preferred organic solvents are the organic amines and alkanolamines. Agents according to the invention preferably contain these amines in amounts of from 0.1 to 10 wt. %, preferably from 0.2 to 8.0 wt. %, and more preferably from 0.5 to 5.0 wt. %, based on the total weight of the agent. Ethanolamine is a particularly preferred alkanolamine.

Agents according to the invention may contain hydrolytic enzymes or other enzymes in a concentration that is expedient for the effectiveness of the agent. Thus, agents further comprising one or more enzymes represent a further embodiment of the invention. All enzymes which can develop catalytic activity in the agent according to the invention, in particular selected from proteases, lipases, amylases, cellulases, hemicellulases, mannanases, tannanases, xylanases, xanthanases, xyloglucanases, β-glucosidases, pectinases, carrageenanases, perhydrolases, oxidases, or oxidoreductases, and mixtures thereof, can preferably be used as enzymes. Enzymes are advantageously contained in the agent in an amount of from 1×10⁻⁸ to 5 wt. % based on active protein and the total weight of the agent. Increasingly preferably, each enzyme is contained in agents according to the invention in an amount of from 1×10⁻⁷ to 3 wt. %, from 0.00001 to 1 wt. %, from 0.00005 to 0.5 wt. %, from 0.0001 to 0.1 wt. %, and particularly preferably from 0.0001 to 0.05 wt. %, based on active protein and the total weight of the agent. Particularly preferably, enzymes exhibit synergistic cleaning performance with respect to particular dirt or stains, i.e., the enzymes contained in the agent composition assist one another in their cleaning performance.

In preferred embodiments, an agent according to the invention contains at least one enzyme and increasingly preferably at least two, three, four or five enzymes, preferably selected from the group consisting of amylases, proteases, lipases, cellulases, mannanases and mixtures thereof, in a total amount of from 0.01 to 10 wt. %, preferably from 0.1 to 8 wt. %, more preferably from 0.2 to 6 wt. %, based on active protein and the total weight of the agent.

Examples of proteases are the subtilisins BPN′ from Bacillus amyloliquefaciens and Carlsberg from Bacillus licheniformis, protease PB92, subtilisins 147 and 309, the protease from Bacillus lentus, subtilisin DY, and the enzymes thermitase, proteinase K and proteases TW3 and TW7, which in the narrower sense are associated with the subtilases but no longer with the subtilisins. Subtilisin Carlsberg is available in a developed form under the trade name Alcalase® from Novozymes. Subtilisins 147 and 309 are marketed by Novozymes under the trade names Esperase® and Savinase®, respectively. Protease variants, described, for example, in WO 95/23221, WO 92/21760, WO 2013/060621 and EP 3660151 are derived from the protease from Bacillus lentus DSM 5483. Other proteases that are suitable are, for example, the enzymes available under the trade names Durazym®, Relase®, Everlase®, Nafizym®, Natalase®, Kannase®, Progress Uno 101L® and Ovozyme® from Novozymes, the enzymes available under the trade names Purafect®, Purafect® OxP, Purafect® Prime, Excellase® and Properase®, Preferenz P100® and Preferenz P300® from Danisco/DuPont, the enzyme available under the trade name Lavergy pro 104 LS® from BASF, the enzyme available under the trade name Protosol® from Advanced Biochemicals Ltd., the enzyme available under the trade name Wuxi® from Wuxi Snyder Bioproducts Ltd., the enzymes available under the trade names Proleather® and Protease P® from Amano Pharmaceuticals Ltd., and the enzyme available under the name Proteinase K-16 from Kao Corp. The proteases from Bacillus gibsonii and Bacillus pumilus, which are disclosed in WO 2008/086916, WO 2007/131656, WO 2017/215925, WO 2021/175696 and WO 2021/175697, are particularly preferably used.

Examples of amylases are the α-amylases from Bacillus licheniformis, Bacillus amyloliquefaciens or Bacillus stearothermophilus, and, in particular, their further developments improved for use in detergents and cleaning agents. The enzyme from Bacillus licheniformis is available from Novozymes under the name Termamyl® and from Danisco/DuPont under the name Purastar® ST. Development products of this α-amylase are available under the trade names Duramyl® and Termamyl® ultra (both from Novozymes), Purastar® OxAm (Danisco/DuPont) and Keistase® (Daiwa Seiko Inc.). The α-amylase from Bacillus amyloliquefaciens is marketed by Novozymes under the name BAN®, and derived variants from the α-amylase from Bacillus stearothermophilus are marketed under the names BSG® and Novamyl®, also by Novozymes. Others that are particularly noteworthy for this purpose are the α-amylases from Bacillus sp. A 7-7 (DSM 12368) and the cyclodextrin glucanotransferase (CGTase) from Bacillus agaradherens (DSM 9948) should be emphasized. Fusion products of all mentioned molecules can also be used. Furthermore, the developments of the α-amylase from Aspergillus niger and A. oryzae, available under the trade name Fungamyl® from Novozymes, are suitable. Other commercial products that can be advantageously used are, for example, Amylase-LT® and Stainzyme® or Stainzyme® ultra or Stainzyme® plus as well as Amplify™ 12 L or Amplify Prime® 100 L or Amplify Prime™ 120 L, the latter also from Novozymes, and the PREFERENZ S® series from Danisco/DuPont, comprising, for example, PREFERENCE S100®, PREFERENCE S1000® or PREFERENCE S210®. Variants of these enzymes that can be obtained by point mutations may also be used.

Suitable cellulases include those of bacterial or fungal origin. Chemically modified or protein-engineered mutants are included. Suitable cellulases are cellulases from the genera Bacillus, Pseudomonas, Humicola, Fusarium, Thielvia, Acremonium, e.g., the fungal cellulase from Humicola insolens, Mycelophthora thermophila and Fusarium oxysporum, which are disclosed in U.S. Pat. Nos. 4,435,307, 5,648,263, 5,691,178, 5,776,757 and WO 89/09259. Particularly suitable cellulases are the alkaline or neutral cellulases with color care properties. Examples of such cellulases are cellulases which are described in EP 0495257, EP 0531372, WO 96/11262, WO 96/29397 and WO 98/08940. Other examples are cellulase variants as described in WO 94/07998, EP 0531315, EP 3212777, EP 3502243, EP 3653705, EP 3653706, U.S. Pat. Nos. 5,457,046, 5,686,593, 5,763,254, WO 95/24471, WO 98/12307 and WO 99/01544 and WO 2019/122520. Examples of cellulases with endo-1,4-glucanase activity (EC 3.2.1.4) are described in WO 2002/099091, for example those having a sequence of at least 97% identity to the amino acid sequence of positions 1 to 773 of SEQ ID NO:2 of WO 2002/099091. A further example can comprise a GH44-xyloglucanase, for example a xyloglucanase enzyme having a sequence of at least 60% identity to positions 40 to 559 of SEQ ID NO:2 of WO 2001/062903. Other examples of cellulases comprise the GH45 cellulases described in WO 96/29397. The commercially available cellulases include Celluzyme™, Carezyme™, Carezyme Premium™, Celluclean™ (e.g., Celluclean™ 5000 L to Celluclean™ 4000T), Celluclean Classic™, Cellusoft™, Endolase®, Renozyme® and Whitezyme™ (Novozymes A/S), Clazinase™ and Puradax HA™ (Genencor International Inc.), KAC-500 (B)™ (Kao Corporation), Revitalenz™ 1000, Revitalenz™ 2000 and Revitalenz® 3000 (DuPont), as well as Ecostone® and Biotouch® (AB Enzymes).

Suitable lipases are, for example, from Thermomyces, e.g., from T. lanuginosus (formerly Humicola lanuginosa) as described in EP 0258068 and EP 0305216, lipase from strains of Pseudomonas (some of them now renamed Burkholderia), e.g., P. alcaligenes or P. pseudoalcaligenes, P. cepacia, P. sp. strain SD705, P. wisconsinensis, Streptomyces lipases of the GDSL type, lipase from Thermobifida fusca, lipase from Geobacillus stearothermophilus, lipase from Bacillus subtilis and lipase from Streptomyces griseus and S. pristinaespiralis. The lipases that can originally be obtained from Humicola lanuginosa (Thermomyces lanuginosus) or have been developed therefrom, in particular those having one or more of the following amino acid exchanges in positions D96 L, T213R and/or N233R, particularly preferably T213R and N233R, proceeding from the mentioned lipase, belong to the preferred lipases. Preferred commercial lipase products include Lipolase™, Lipex™, Lipolex™ and Lipoclean™ (Novozymes A/S), Lumafast (Genencor/DuPont) and Lipomax (Gist-Brocades).

Suitable mannanases are, for example, Bacillus subtilis endo-β-mannanase, Bacillus sp. 1633 endo-β-mannanase, Bacillus sp. AAI12 endo-β-mannanase, Bacillus sp. AA349 endo-β-mannanase, Bacillus agaradhaerens NCIMB 40482 endo-β-mannanase, Bacillus halodurans endo-β-mannanase, Bacillus clausii endo-β-mannanase, Bacillus licheniformis endo-β-mannanase, Humicola insolens endo-β-mannanase and Caldicellulosiruptor sp. endo-β-mannanase (e.g., U.S. Pat. No. 6,060,299, WO 99/64573, U.S. Pat. No. 6,566,114 and WO 99/64619).

Pectate lyases suitable for detergents and cleaning agents are described, for example, in WO 2003/095638 or WO 2015/121133. Examples of suitable pectinolytic enzymes are also the enzymes and enzyme preparations available under the trade names Gamanase®, Pektinex AR®, X-Pect® or Pectaway® from Novozymes, under the trade names Rohapect UF®, Rohapect TPL®, Rohapect PTE100®, Rohapect MPE®, Rohapect MA plus HC, Rohapect DA12L®, Rohapect 10L®, Rohapect B1L® from AB Enzymes, and under the trade name Pyrolase® from Diversa Corp.

The peptides, proteins and enzymes described herein are preferably mature peptides, proteins or enzymes, i.e., the catalytically active molecule without signal and/or propeptide(s). Unless otherwise stated, the sequences given also refer to respective mature (processed) peptides, proteins or enzymes.

In various embodiments of the invention, the respective peptide, protein or enzyme is a free peptide, protein or enzyme. This means that the peptide, protein or enzyme can interact directly with all components of the agent and, if the agent is a liquid agent, that the peptide, protein or enzyme is in direct contact with the solvent of the agent (e.g., water). In other embodiments, an agent may contain peptides, proteins or enzymes that form an interaction complex with other molecules or that contain an “envelope”. In this case, a single peptide, protein or enzyme molecule, or a plurality thereof, can be separated from the other components of the agent by a surrounding structure. Such a separating structure can arise due to, but is not limited to, vesicles, such as a micelle or a liposome. However, the surrounding structure may also be a virus particle, a bacterial cell or a eukaryotic cell. In various embodiments, an agent may contain cells of Bacillus pumilus or Bacillus subtilis which express peptides, proteins or enzymes, or cell culture supernatants of such cells.

In the context of the present invention, the feature means that a peptide, protein or enzyme has the indicated substitution(s) (or deletion or insertion), that it contains a (the indicated) substitution(s) (or deletion or insertion) at the corresponding position, i.e., at least the indicated positions are not otherwise mutated or deleted, e.g., by fragmentation of the peptide, protein or enzyme. In various embodiments, the peptides, proteins and/or enzymes described herein have the sequence of the respective reference sequence except for the explicitly mentioned substitutions, i.e., are 100% identical to the respective reference sequence except for the substituted positions.

The identity of nucleic acid or amino acid sequences is determined by a sequence comparison. This sequence comparison is based on the BLAST algorithm established and commonly used in the prior art (Altschul et al., Basic local alignment search tool, J. Mol. Biol., 1990, 215, 403-410, and Altschul et al., Gapped BLAST and PSI-BLAST: a new generation of protein database search programs, Nucleic Acids Res., 1997, 25, 3389-3402) and occurs in principle by similar sequences of nucleotides or amino acids in the nucleic acid or amino acid sequences being assigned to one another. A tabular assignment of the relevant positions is referred to as an alignment. A further algorithm available in the prior art is the FASTA algorithm. Sequence comparisons (alignments), in particular multiple sequence comparisons, are created using computer programs. Frequently used are, for example, the Clustal series (Chenna et al., Multiple sequence alignment with the Clustal series of programs, Nucleic Acid Res., 2003, 31, 3497-3500), T-Coffee (Notredame et al., T-Coffee: A novel method for multiple sequence alignments, J. Mol. Biol., 2000, 302, 205-217) or programs based on these programs or algorithms. Also possible are sequence comparisons (alignments) using the computer program Vector NTI® Suite 10.3 (Invitrogen Corporation, 1600 Faraday Avenue, Carlsbad, California, USA) with the specified standard parameters, the AlignX module of which for the sequence comparisons is based on ClustalW, or Clone Manager 10 (use of the scoring matrix BLOSUM 62 fir sequence alignment at amino acid level). Unless stated otherwise, the sequence identity indicated herein is determined using the BLAST algorithm.

Such a comparison also allows a conclusion to be drawn about the similarity of the compared sequences to one another. It is usually given in percent identity, i.e., the proportion of identical nucleotides or amino acid residues at the same positions or positions corresponding to one another in an alignment. In the case of amino acid sequences, the broader concept of homology takes conserved amino acid exchanges into account, i.e., amino acids having similar chemical activity, as these usually perform similar chemical activities within the peptide, protein or enzyme. Therefore, the similarity of the compared sequences can also be indicated as percent homology or percent similarity. Identity and/or homology information can be provided regarding whole polypeptides or genes or only regarding individual regions. Homologous or identical regions of different nucleic acid or amino acid sequences are therefore defined by matches in the sequences. Such regions often have identical functions. They can be small and comprise only a few nucleotides or amino acids. Such small regions often perform essential functions for the overall activity of the peptide, protein or enzyme. It may therefore be expedient to relate sequence matches only to individual, optionally small, regions. Unless otherwise stated, however, identity or homology information in the present application relates to the entire length of the particular nucleic acid or amino acid sequence indicated.

In the context of the present invention, the indication that an amino acid position corresponds to a numerically designated position in SEQ ID NO:1 therefore means that the corresponding position is associated with the numerically designated position in SEQ ID NO:1 in an alignment as defined above.

For the description of substitutions that relate to exactly one amino acid position (amino acid exchanges), the following convention is applied herein: first, the naturally present amino acid is referred to in the form of the internationally used single-letter code, followed by the associated sequence position and finally the inserted amino acid. Several or alternative exchanges within the same polypeptide chain are separated by slashes. “130D/V” thus means that position 130 has mutated to D or V. In the case of insertions, additional amino acids are named according to the sequence position. In the case of deletions, the missing amino acid is replaced by a symbol, for example a star or a dash, or a Δ is indicated before the corresponding position. For example, P9T describes the substitution of proline at position 9 by threonine, P9TH describes the insertion of histidine following the amino acid threonine at position 9 and P9* or ΔP9 describes the deletion of proline at position 9. This nomenclature is known to a person skilled in the art in the field of enzyme technology.

It is possible for a person skilled in the art to use methods which are currently generally known, for example chemical synthesis or polymerase chain reaction (PCR), in conjunction with standard molecular biology and/or protein-chemical methods, to produce the corresponding nucleic acids and even complete genes using known DNA and/or amino acid sequences. Such methods are known, for example, from Sambrook, J., Fritsch, E. F. and Maniatis, T. 2001. Molecular cloning: a laboratory manual, 3. Edition Cold Spring Laboratory Press.

Furthermore, the peptides, proteins, enzymes and/or other ingredients of the agent that are contained therein can be coated with a substance which is impermeable to the peptide, protein or enzyme at room temperature or in the absence of water and which becomes permeable to the peptide, protein or enzyme under the conditions of use of the agent. Such an embodiment of the invention is thus characterized in that the peptide, protein or enzyme is coated with a substance which is impermeable to the peptide, protein or enzyme at room temperature or in the absence of water. Furthermore, the detergent or cleaning agent itself can also be packaged in a container, preferably an air-permeable container, from which it is released shortly before use or during the washing process.

In the agents described herein, the peptides, proteins or enzymes to be used may further be packaged together with accompanying substances, for example from fermentation. In liquid formulations, the peptides, protein or enzymes are preferably used as peptide, protein or enzyme liquid formulation(s).

The peptides, protein or enzymes are generally not provided in the form of pure protein, but rather in the form of stabilized, storable and transportable preparations. These pre-packaged preparations include, for example, the solid preparations obtained by granulation, extrusion, or lyophilization or, in particular in the case of liquid or gel agents, solutions of the peptides, proteins or enzymes, which are advantageously as concentrated as possible, have a low water content, and/or are supplemented with stabilizers or other auxiliaries.

Alternatively, the peptides, proteins or enzymes can also be encapsulated for both the solid and the liquid dosage forms, e.g., by spray drying or extrusion of the peptide, protein or enzyme solution together with a preferably natural polymer or in the form of capsules, e.g., those in which the peptides, proteins or enzymes are encapsulated as in a solidified gel, or in those of the core-shell type in which an enzyme-containing core is coated with a protective layer that is impermeable to water, air and/or chemicals. Other active ingredients such as stabilizers, emulsifiers, pigments, bleaching agents, or dyes can additionally be applied in overlaid layers. Such capsules are applied using methods that are known per se, for example by shaking or roll granulation or in fluidized bed processes. Advantageously, such granules are low in dust, for example due to the application of polymeric film-formers, and stable in storage due to the coating.

Furthermore, it is possible to package two or more peptides, proteins or enzymes together such that a single granule comprises a plurality of enzyme activities.

The peptides, proteins or enzymes can also be introduced into water-soluble films, such as those used in the packaging of detergent and cleaning agents in a unit dosage form. Such a film enables the release of peptides, proteins or enzymes after contact with water. As used herein, “water-soluble” refers to a film structure that is preferably completely water-soluble. Preferably, such a film consists of (completely or partially hydrolyzed) polyvinyl alcohol (PVA).

Agents according to the invention may comprise one or more reversible enzyme inhibitor(s)/stabilizer(s). Agents according to the invention may contain the reversible enzyme inhibitor(s)/stabilizer(s) in a concentration of 0.1 to 2 wt. %, preferably 0.3 to 1.5 wt. %, based on the total weight of the agent. If several inhibitors/stabilizers are contained, this information refers to the total concentration. These may in particular be selected from the group consisting of polyols, such as glycerol or 1,2-ethylene glycol, benzamidine hydrochloride, borax, boric acids, boronic acids or their salts or esters or derivatives, in particular phenylboronic acid derivatives or 4-formylphenylboronic acid (4-FPBA), antioxidants, special peptide compounds and combinations thereof.

In the context of the present invention, “phenylboronic acid derivative” is understood to mean a compound of the following formula:

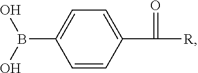

where R is hydrogen, a hydroxyl group, a C₁₋₆ alkyl group, a substituted C₁₋₆ alkyl group, a C₁₋₆ alkenyl group or a substituted C₁₋₆ alkenyl group. Preferably, the functional group R in the phenylboronic acid derivative is a C₁₋₆ alkyl group and more preferably —CH₃, —CH₃CH₂ or —CH₃CH₂CH₂. More preferably, the functional group R in the phenylboronic acid derivative is hydrogen. The phenylboronic acid derivative 4-formylphenylboronic acid (4-FPBA) is particularly preferred.

The inhibitor/stabilizer compound used can be boric acid.

In preferred embodiments, the agent according to the invention is substantially free of boron-containing compounds. “Substantially free from boron-containing compounds” in this context means that the agents according to the invention contain less than 2 wt. %, preferably less than 1 wt. %, more preferably less than 0.5 wt. %, and particularly preferably less than 0.1 wt. %, of boron-containing compounds, based on the total weight of the agent. In particularly preferred embodiments, the agents according to the invention are free of boron-containing compounds, i.e., in particular they do not contain any boric acid and/or phenylboronic acid derivatives.

In various embodiments, the enzyme and the inhibitor/stabilizer compound may be pre-formulated in an enzyme composition. As is apparent from the previous statements, the enzyme protein forms only a fraction of the total weight of conventional enzyme preparations. Enzyme preparations that are preferably used contain between 0.1 and 40 wt. %, preferably between 0.2 and 30 wt. %, particularly preferably between 0.4 and 20 wt. %, and in particular between 0.8 and 10 wt. % of the enzyme protein. In such compositions, the inhibitor/stabilizer compound can be contained in an amount of from 0.05 to 35 wt. %, preferably from 0.05 to 10 wt. %, based on the total weight in the enzyme composition. This enzyme composition can be used in agents according to the invention, in amounts leading to the final concentrations in the agent as indicated above.

In preferred embodiments, the invention relates to an agent for cleaning textiles and/or hard surfaces, in particular dishes, characterized in that the agent is a detergent and cleaning agent, preferably a liquid detergent and cleaning agent, preferably a liquid textile detergent, and comprises
(A) at least one peptide, wherein the peptide
   has a length of 10 to 50, preferably 10 to 30, preferably 12 to 25, amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, preferably selected from alanine, arginine, glutamic acid, glycine, isoleucine, leucine, lysine, phenylalanine, proline, serine, threonine, tryptophan, tyrosine and valine,
   has a net charge of +2 to +8, preferably +3 to +7, preferably +4 to +6,
   is antimicrobially active, the antimicrobial activity being determined as described in Example 1,
   preferably in an amount of active peptide of 1×10⁻⁸ to 5 wt. %, preferably 0.0001 to 1 wt. %, preferably 0.0005 to 0.5 wt. %, particularly preferably 0.001 to 0.1 wt. %, based on the total weight of the agent;
(B) at least one surfactant, preferably in an amount of 3 to 35 wt. %, preferably 5 to 30 wt. %, based on the total weight of the agent, wherein the surfactant is selected from the group consisting of anionic surfactants, non-ionic surfactants, cationic surfactants, zwitterionic surfactants, amphoteric surfactants and mixtures thereof;
(C) a builder system, comprising at least one builder, preferably in an amount of 0.5 to 50 wt. %, preferably 0.5 to 20 wt. %, particularly preferably 0.5 to 10 wt. %., based on the total weight of the agent, wherein the builder is selected from the group consisting of polycarboxylic acids such as citric acid, adipic acid, succinic acid, glutaric acid, malic acid, tartaric acid, maleic acid, fumaric acid, saccharic acids and carboxymethyl inulines or salts thereof, monomeric and polymeric aminopolycarboxylic acids such as glycine diacetic acid, methylglycine diacetic acid (MGDA), glutamic acid diacetic acid (GLDA), nitrile triacetic acid, iminodisuccinate such as ethylenediamine-N,N′-disuccinic acid and hydroxyiminodisuccinates, ethylenediaminetetraacetic acid and polyaspartic acid or salts thereof, polyphosphonic acids such as aminotris(methylenephosphonic acid), ethylenediaminetetrakis(methylenephosphonic acid), lysinetetra(methylenephosphonic acid), diethylenetriaminepenta(methylenephosphonic acid) (DTPMP) and 1-hydroxyethane-1,1-diphosphonic acid (HEDP) or salts thereof, polymeric hydroxy compounds such as dextrin, and mixtures thereof; and
(D) optionally at least one enzyme, preferably in an amount of 1×10⁻⁸ to 5 wt. %, preferably 0.0001 to 1 wt. %, preferably 0.0005 to 0.5 wt. %, particularly preferably 0.001 to 0.1 wt. %, based on active protein and total weight of the agent, wherein the enzyme is selected from the group consisting of proteases, amylases, lipases, mannanases, cellulases or mixtures thereof.

In a preferred embodiment, the invention relates to an agent for cleaning surfaces, in particular household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets, and/or textiles, in particular for preventing and/or reducing bad odors and/or for improving hygiene, characterized in that the agent is a detergent and cleaning agent, preferably a liquid detergent and cleaning agent, preferably a liquid textile detergent, and comprises
(A) at least one peptide, wherein the peptide
   has a length of 10 to 50, preferably 10 to 30, preferably 12 to 25, amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, preferably selected from alanine, arginine, glutamic acid, glycine, isoleucine, leucine, lysine, phenylalanine, proline, serine, threonine, tryptophan, tyrosine and valine,
   has a net charge of +2 to +8, preferably +3 to +7, preferably +4 to +6,
   is antimicrobially active, the antimicrobial activity being determined as described in Example 1,
   preferably in an amount of active peptide of 1×10⁻⁸ to 5 wt. %, preferably 0.0001 to 1 wt. %, preferably 0.0005 to 0.5 wt. %, particularly preferably 0.001 to 0.1 wt. %, particularly preferably 0.001 to 0.25 wt. %, based on the total weight of the agent;
(B) at least one surfactant, preferably in an amount of 3 to 35 wt. %, preferably 5 to 30 wt. %, based on the total weight of the agent, wherein the surfactant is selected from the group consisting of anionic surfactants, non-ionic surfactants, cationic surfactants, zwitterionic surfactants, amphoteric surfactants and mixtures thereof;
(C) a builder system, comprising at least one builder, preferably in an amount of 0.5 to 50 wt. %, preferably 0.5 to 20 wt. %, particularly preferably 0.5 to 10 wt. %., based on the total weight of the agent, wherein the builder is selected from the group consisting of polycarboxylic acids such as citric acid, adipic acid, succinic acid, glutaric acid, malic acid, tartaric acid, maleic acid, fumaric acid, saccharic acids and carboxymethyl inulines or salts thereof, monomeric and polymeric aminopolycarboxylic acids such as glycine diacetic acid, methylglycine diacetic acid (MGDA), glutamic acid diacetic acid (GLDA), nitrile triacetic acid, iminodisuccinate such as ethylenediamine-N,N′-disuccinic acid and hydroxyiminodisuccinates, ethylenediaminetetraacetic acid and polyaspartic acid or salts thereof, polyphosphonic acids such as aminotris(methylenephosphonic acid), ethylenediaminetetrakis(methylenephosphonic acid), lysinetetra(methylenephosphonic acid), diethylenetriaminepenta(methylenephosphonic acid) (DTPMP) and 1-hydroxyethane-1,1-diphosphonic acid (HEDP) or salts thereof, polymeric hydroxy compounds such as dextrin, and mixtures thereof; and
(D) optionally at least one enzyme, preferably in an amount of 1×10⁻⁸ to 5 wt. %, preferably 0.0001 to 1 wt. %, preferably 0.0005 to 0.5 wt. %, particularly preferably 0.001 to 0.1 wt. %, based on active protein and total weight of the agent, wherein the enzyme is selected from the group consisting of proteases, amylases, lipases, mannanases, cellulases or mixtures thereof.

This also comprises respective agents for (semi-)automated washing or cleaning systems such as floor-mopping robots or wet vacuum cleaners.

All facts, objects and embodiments described for peptides according to the invention and agents according to the invention are also applicable to these objects of the invention. Therefore, reference is expressly made at this point to the disclosure at the corresponding point with the indication that this disclosure also applies to the above use according to the invention.

A further object of the invention also relates to cosmetic agents, such as washing and care products for hair care, and industrial and institutional cleaners, such as disinfectant products, in particular disinfectants, wherein such agents contain an antimicrobial peptide described herein.

In further preferred embodiments, the invention relates to
a method for cleaning textiles and/or hard surfaces, in particular dishes, characterized in that a detergent and cleaning agent described herein is used in at least one method step, the method preferably being carried out in a temperature range from about 20° C. to about 60° C., preferably about 40° C., particularly preferably about 30° C.; and
a method for cleaning surfaces, in particular household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets, and/or textiles, in particular for preventing and/or reducing bad odors and/or for improving hygiene, characterized in that a detergent and cleaning agent described herein is used in at least one method step, the method preferably being carried out in a temperature range from about 20° C. to about 60° C., preferably about 40° C., particularly preferably about 30° C.

This includes both manual and machine methods, with machine methods being preferred because they can be controlled more precisely, for example with regard to the quantities used and contact times. Methods for cleaning textiles are generally characterized in that various cleaning-active substances are applied to the items to be cleaned in a plurality of method steps and washed off after the exposure time, or in that the items to be cleaned are treated in some other way with an agent or a solution or dilution of said agent. This also comprises methods using (semi)automated washing or cleaning systems such as floor-mopping robots or wet vacuum cleaners.

This object of the invention also comprises a dishwashing machine method. The cleaning agent according to the invention can be dispensed into the cleaning liquor in such a method for example by means of the dispensing chamber in the door or by means of an additional dispensing container in the interior of the dishwasher. Alternatively, the cleaning agent can also be applied directly to the dirty dishes or to one of the interior walls of the dishwasher, for example the inside of the door. The method according to the invention is carried out in the interior of a commercially available dishwasher. In the case of a dishwasher, the cleaning program can generally be selected and determined by the user before the dishwashing method is carried out. The dishwasher cleaning program used in the method according to the invention comprises at least one prewash cycle and one cleaning cycle. Cleaning programs which comprise further cleaning or rinsing cycles, e.g., a rinse cycle, are preferred according to the invention. The method according to the invention is particularly preferably part of a cleaning program comprising a prewash cycle, a cleaning cycle and a rinse cycle. The method according to the invention is preferably used in connection with cleaning programs in which the washing liquor is heated during the cleaning cycle. In a preferred embodiment of the method according to the invention, the cleaning cycle during which the cleaning agent according to the invention is dispensed into the interior of the dishwasher is characterized in that the temperature of the cleaning liquor during said cycle rises to values above 30° C., preferably above 40° C. and in particular above 50° C.

Alternative embodiments of this subject matter of the invention are also represented by methods for treating textile raw materials or for textile care, in which an agent according to the invention becomes active in at least one method step. Among these, methods for textile raw materials, fibers or textiles comprising natural constituents are preferred, and very particularly for those comprising wool or silk.

All facts, objects and embodiments described for peptides according to the invention and agents according to the invention are also applicable to these objects of the invention. Therefore, reference is expressly made at this point to the disclosure at the corresponding point with the indication that this disclosure also applies to the above use according to the invention.

In further preferred embodiments, the invention relates to
a use of a peptide in a detergent and cleaning agent for cleaning textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets, preferably in a temperature range from about 20° C. to about 60° C., preferably about 40° C., particularly preferably about 30° C., the peptide being as defined herein;
a use of a peptide in a detergent and cleaning agent for reducing bad odors from textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets which have been treated with the agent, preferably in a temperature range from about 20° C. to about 60° C., preferably about 40° C., particularly preferably about 30° C., the peptide being as defined herein;
a use of a peptide in a detergent and cleaning agent for disinfecting textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets which have been treated with the agent, preferably in a temperature range from about 20° C. to about 60° C., preferably about 40° C., particularly preferably about 30° C., the peptide being as defined herein; and
a use of a peptide in a detergent and cleaning agent for preventing and/or reducing microbial grown on textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets which have been treated with the agent, preferably in a temperature range from about 20° C. to about 60° C., preferably about 40° C., particularly preferably about 30° C., the peptide being as defined herein.

All facts, objects and embodiments described for peptides according to the invention and agents according to the invention are also applicable to these objects of the invention. Therefore, reference is expressly made at this point to the disclosure at the corresponding point with the indication that this disclosure also applies to the above use according to the invention.

### EXAMPLES

### Example 1: Determination of Antimicrobial Activity

TABLE 1
DETERGENT MATRIX USED

| | Wt. % of active | Wt. % of active substance | |
|---|---|---|---|
| | substance in the | in the formulation | |
| Chemical name | raw material | A | B |
| Demineralized water | 100 | Remainder | Remainder |
| 1,2-propanediol | 100 | 5.0 | 5.0 |
| Glycerol | 99.5 | 9.4 | 9.4 |
| Monoethanolamine | 100 | 6.0 | 6.0 |
| Alkyl benzene sulfonic acid | 96 | 21.7 | 21.7 |
| Palm kernel oil | 100 | 7.0 | 7.0 |
| C₁₂₋₁₈ fatty alcohol ethoxylate, 7EO | 100 | 22.4 | 22.4 |
| Ethoxylated polyethyleneimine | 80 | 4.5 | 4.5 |
| DTPMP-Na7 | 40 | 0.5 | 0.5 |
| Enzymes | t.q. | 0 | Minors |
| Misc. (SRP, DTI, perfume, opt. brighteners, | t.q. | minors | minors |
| bitterns, antifoam) | | | |

Dosage 3.17 g/L; pH 8.2-8.4

TABLE 2
PEPTIDES USED

| | | SEQ | |
|---|---|---|---|
| Peptide | Amino acid | ID | |
| number | sequence | NO: | Source |
| AMP 1 | ILRWPWWPWRRK | 1 | Sader et al.¹ |
| AMP 2 | FSTKTRNWFSEHF | 2 | Barksdale et al.² |
| | KKVKEKLKDTFA | | |
| AMP 3 | KTRNWFSEHFKKV | 3 | Barksdale et al.² |
| | KEKLKDTFA | | |
| AMP 4 | WRWAKWGLKLLKY | 4 | Fleeman et al.³ |
| | KKIY | | |
| AMP 5 | IWWAKWGLKLLRY | 5 | Fleeman et al.³ |
| | RRWY | | |

¹Sader et al. (2004), Omiganan Pentahydrochloride (MBI 226), a Topical 12-Amino-Acid Cationic Peptide: Spectrum of Antimicrobial Activity and Measurements of Bactericidal Activity, Antimicrobial Agents and Chemotherapy, 48 (8): 3112-3118;
²Barksdale et al. (2016) Peptides from American alligator plasma are antimicrobial against multi-drug resistant bacterial pathogens including Acinetobacter baumannii, BMC Microbiology, 16: 189-202;
³Fleeman et al. (2020) Defining principles that influence antimicrobial peptide activity against capsulated Klebsiella pneumoniae, PNAS, 117 (44): 27620-27626

The antibacterial activity of the peptides shown in Table 2 was tested at a concentration of 5,000 mg/L in an MPN suspension test (MPN=Most Probable Number) on various microorganisms (Staphylococcus aureus ATCC 6538/K3212, initial bacterial count 3.6E±0.6; Klebsiella pneumoniae ATCC 4352/K2510, initial bacterial count 6.1E±06). The respective peptide solutions were dissolved in water (control), detergent without enzyme (A) and detergent with enzyme (B) at a concentration of 5,000 mg/L.

The MPN suspension test was performed in deep-well microtiter plates (1,000 μl volume per well), each well containing 800 μl application concentration (AWK, prepared in distilled water) with a 1.25-fold concentration of the test substance (final concentration: 5,000 mg/L). In each case, 100 μl of the bacterial suspension was added (final concentration: 1.0E±08 CFU/ml). To homogenize the test preparations, the microtiter plates were placed on a Titramax during the test. A water control was carried out parallel to the test preparations. Instead of the test substance, dist. water was used.

After incubation for 60 min at 30° C., an aliquot of the test preparations and the water control were inoculated into an inactivation solution. For the inactivation step (1:10 dilution), 100 μl test mixture was mixed with 900 μl nutrient broth (containing inactivation agent) (final concentration: 1.0E±06 CFU/ml in the inactivation preparation).

After at least 5 min and a maximum of 30 min inactivation, the number of living test germ cells was determined. Decadal dilution series were prepared in 96-well microtiter plates (200 μl volume per well), in which 180 μl nutrient broth (containing inactivating agent) was added per well. The dilution series were carried out in three parallels, i.e., three wells from each of the inactivation preparations (=test mixture plus inactivating agent in nutrient broth) were inoculated and diluted decadically. For each well, 20 μl of inactivated preparation+180 μl of nutrient broth (containing inactivating agents) were mixed in 5 decadic dilution steps. The covered microtiter plates were then incubated at 37° C. for 48 hours.

The result calculation regarding the killing rates of the test germs used was carried out depending on the water control. For evaluation, the growth in the wells, indicated by turbidity of the nutrient broth, was determined visually or using the Tecan reader.

The microbicidal activity was calculated using the following formula:

RF=K/D or log 10K−log10D,

where RF=reduction factor, K=number of CFU/ml without exposure to the product (water control) and D=number of CFU/ml after exposure to the product.

The results are shown in Table 3.

TABLE 3
ANTIMICROBIAL ACTIVITY AGAINST STAPHYLOCOCCUS AUREUS
AND KLEBSIELLA PNEUMONIAE

| S. aureus (ATCC 6538/K3212) | | Log reduction | | |
|---|---|---|---|---|
| Active ingredient | Concentration | Water | Detergent A | Detergent B |
| AMP 1 | 5000 mg/L | >4.65 | 2.40 | 1.48 |
| AMP 2 | 5000 mg/L | >4.65 | 1.48 | 2.40 |
| AMP 4 | 5000 mg/L | >4.65 | 2.40 | 1.48 |
| Benzalkonium chloride | 100 mg/L | >4.65 | no | no |
| (positive control) | | | reduction | reduction |
| K. pneumoniae (ATCC 4352/K2510) | | Log reduction | | |
| Active ingredient | Concentration | Water | Detergent A | Detergent B |
| AMP 1 | 5000 mg/L | >4.40 | >4.65 | >4.40 |
| AMP 2 | 5000 mg/L | >4.40 | >4.65 | >4.40 |
| AMP 4 | 5000 mg/L | >4.40 | >4.04 | >4.65 |
| Benzalkonium chloride | 100 mg/L | >4.40 | no | no |
| (positive control) | | | reduction | reduction |

The results show that the tested peptides have an antimicrobial effect against both Staphylococcus aureus (Gram-positive bacterium) and Klebsiella pneumoniae (Gram-negative bacterium) and that the number of living cells is significantly reduced, in some cases by up to 4 log levels.

### Example 2: Example Formulations

The peptides according to the invention can be used in various detergents and cleaning agents and can have an antimicrobial effect.

TABLE 4
LIQUID DETERGENTS

| | Wt. % of active substance in the formulation | | |
|---|---|---|---|
| Chemical name | A | B | C |
| Boric acid | 0.6 | 0.6 | 0.6 |
| Citric acid | 0.3 | 0.3 | 0.3 |
| FAEOS | 3.9 | 3.9 | 3.9 |
| FAEO | 4.3 | 4.3 | 4.3 |
| LAS | 3.5 | 3.5 | 3.5 |
| Soap (palm kernel oil) | 0.6 | 0.6 | 0.6 |
| NaOH | 0.8 | 0.8 | 0.8 |
| 1,2-propanediol | 0 | 0 | 0 |
| Glycerol | 0.6 | 0.6 | 0.6 |
| DTPMP | 0.3 | 0 | 0.13 |
| GLDA | 0 | 0.3 | 0.13 |
| Peptide according to the invention | 5000 mg/L | 5000 mg/L | 5000 mg/L |
| Water, demineralized | up to 100 | up to 100 | up to 100 |
| DTI, SRP, other enzymes, defoamers, etc. | minors | minors | minors |

Viscosity [mPas] = 250-550; Density [g/cm³] = 1.03-1.04; pH = 8.2-8.6

TABLE 5
LIQUID DETERGENTS

| | Wt. % of active substance in the formulation | | | | | |
|---|---|---|---|---|---|---|
| Chemical name | A | B | C | D | E | F |
| Demineralized water | Remainder | Remainder | Remainder | Remainder | Remainder | Remainder |
| LAS | 5.5 | 20 | 15.0 | 5.5 | 21.7 | 23.5 |
| FAEOS | 7.0 | | 5.0 | | | |
| Palm kernel oleic acid | 3.0 | 8.0 | | | 7.0 | 7.4 |
| FAEO | 5.5 | | 8.0 | | | |
| C_{13/15} oxo alcohol, 8EO | | 25 | | | | |
| C₁₂₋₁₈ fatty alcohol ethoxylate, | | | | | 22.4 | 23.4 |
| 7EO | | | | | | |
| Alkyl polyglycoside | | | 4.0 | | | |
| Non-ionic surfactants | | | | 3.1 | | |
| Soap | | | 1.0 | 0.5 | | |
| HEDP | 0.5 | | | | | |
| DTPMPA 7Na | | 1.0 | 1.0 | 0.2 | 0.5 | 1.7 |
| Citric acid | 2.5 | | 3.0 | 0.23 | | |
| NaOH | 3.0 | | | 0.7 | | |
| Glycerol | 3.0 | 5.0 | | 0.5 | 9.4 | 10.2 |
| Ethanol | 1.5 | 3.0 | | | | 3.2 |
| 1,2-propanediol | | 10.0 | 12.0 | | 5.0 | 5.6 |
| Monoethanolamine | | 6.0 | 7.0 | | 6.0 | 6.1 |
| Boric acid | 1.0 | | 1.0 | 0.5 | | |
| Polyalkoxylated alkanolamine | | | | | 4.5 | |
| Ethoxylated polyethyleneimine | | | | | 4.5 | 3.0 |
| Peptides according to the | 5000 mg/L | 5000 mg/L | 5000 mg/L | 5000 mg/L | 5000 mg/L | 5000 mg/L |
| invention | | | | | | |
| DTI, SRP, other enzymes, | minors | minors | minors | minors | minors | minors |
| defoamers, etc. | | | | | | |

TABLE 6
SOLID DETERGENTS

| | Wt. % of active substance in the formulation | | |
|---|---|---|---|
| Chemical name | A | B | C |
| LAS | 12.2 | 12.0 | 10.1 |
| Sodium fatty alcohol sulphate, C₁₂₋₁₈ | 4.2 | | |
| Fatty alcohol, C₁₂₋₁₈, 7 EO | 4.1 | 2.3 | 1.5 |
| Soaps | 0.4 | | |
| Citrate | 2.0 | | |
| Sodium carbonate | 2.4 | 17.9 | 25.1 |
| Builders | 23.0 | 7.0 | 7.6 |
| Phosphonate | 1.2 | 1.1 | 1.2 |
| Polyacrylate | 0.12 | 2.8 | 3.0 |
| Carboxymethylcellulose | 2.3 | 2.0 | 1.1 |
| 2Na2 carbonate 3 H2O2 | 18.5 | 15.8 | |
| TAED | 10.9 | 3.5 | |
| Peptides according to the invention | 5000 mg/L | 5000 mg/L | 5000 mg/L |
| Sodium sulfate, foam inhibitor, optical brighteners, | Remainder | Remainder | Remainder |
| fragrances, other enzymes | | | |

TABLE 7
HAND DISHWASHING DETERGENT

| Chemical name | Wt. % of active substance in the formulation |
|---|---|
| Demineralized water | Remainder |
| FAEOS | 8.8 |
| Cocoamidopropyl betaine | 1.2 |
| Calcium lactate | 1.0 |
| Amylase (Stainzyme 12 L) | 0.8 |
| Perfume | 0.2 |
| Dye | 0.01 |
| Salts | 2.0 |
| Peptides according to the invention | 5000 mg/L |
| pH | 8.0 |

TABLE 8
TWO-PHASE DISHWASHING DETERGENT

| Powder phase (phase A) Active substance content in wt. % (unless otherwise | | |
|---|---|---|
| stated), based on the total weight of the powder phase | A1 | A2 |
| Sodium percarbonate | 13.0 | 15.0 |
| Non-ionic surfactants | 4.0 | 4.0 |
| Sulfonic acid group-containing polymer | 4.0 | 4.0 |
| HEDP (sodium salt) | 6.0 | 6.0 |
| Sodium carbonate (incl. sodium hydrogen carbonate) | 24.0 | 28.0 |
| MGDA (trisodium salt) | 0 | 0 |
| Phyllosilicate (SKS 6 powder) | 4.0 | 4.0 |
| Sodium citrate (calculated as anhydrous sodium citrate) | 21.0 | 21.0 |
| TAED | 2.1 | 2.1 |
| Zinc acetate | 0.15 | 0.15 |
| Amylase (Stainzyme ® Plus 24 Evity T; indication as wt. % | 1.5 | 1.5 |
| based on the amount of preparation used, t.q.) | | |
| Protease (Unity 1000; total active protein) | 40 mg/job | 40 mg/job |
| Peptides according to the invention | 5000 mg/L | 5000 mg/L |
| Silver protection (cysteine) | 0.15 | 0.15 |
| Misc (perfume, dyes, preservatives, fillers e.g., sodium | Add 100 | Add 100 |
| sulfate, bleach catalyst (MnTACN)) | | |
| Gel phase (phase B) Active substance content in wt. % (unless otherwise | | |
| stated), based on the total weight of the gel phase | B1 | B2 |
| Polymer comprising acrylic acid-containing and amidopropyl | 10.0 | 11.0 |
| sulfonic acid-containing monomers | | |
| Glycerol | 27.0 | 25.0 |
| 1,3-propanediol | 30.0 | 30.0 |
| PEG 400 | 15.0 | 17.0 |
| PVOH (POVAL 4-88) | 15.0 | 14.0 |
| Misc (inter alia, process auxiliaries, pH adjusters, perfume, dye) | Add 100 | Add 100 |
| Gelling time/min | less than 1 | less than 1 |

The phases A1 or A2 and the phases B1 or B2 can be combined with one another as desired. Total weight of both phases in a single portion of 18.5 g.

1. An agent, comprising:
(A) at least one peptide, wherein the peptide;
has a length of 10 to 50, 10 to 30, or 12 to 25, amino acids selected from the group consisting of: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, and wherein, alanine, arginine, glutamic acid, glycine, isoleucine, leucine, lysine, phenylalanine, proline, serine, threonine, tryptophan, tyrosine and valine are preferred selections;
has a net charge of +2 to +8, +3 to +7, or 4 to +6; and,
is antimicrobially active, the antimicrobial activity being determined as described in Example 1;
(B) at least one surfactant, wherein the surfactant is selected from the group consisting of: anionic surfactants, non-ionic surfactants, cationic surfactants, zwitterionic surfactants, amphoteric surfactants, and mixtures thereof;
(C) a builder system comprising at least one builder, wherein the builder is selected from the group consisting of: polycarboxylic acids such as citric acid, adipic acid, succinic acid, glutaric acid, malic acid, tartaric acid, maleic acid, fumaric acid, saccharic acids and carboxymethyl inulines or salts thereof, monomeric and polymeric aminopolycarboxylic acids such as glycine diacetic acid, methylglycine diacetic acid (MGDA), glutamic acid diacetic acid (GLDA), nitrile triacetic acid, iminodisuccinate such as ethylenediamine-N,N′-disuccinic acid and hydroxyiminodisuccinates, ethylenediaminetetraacetic acid and polyaspartic acid or salts thereof, polyphosphonic acids such as aminotris(methylenephosphonic acid), ethylenediaminetetrakis(methylenephosphonic acid), lysinetetra(methylenephosphonic acid), diethylenetriaminepenta(methylenephosphonic acid) (DTPMP) and 1-hydroxyethane-1,1-diphosphonic acid (HEDP) or salts thereof, polymeric hydroxy compounds such as dextrin, and mixtures thereof; and,
(D) optionally at least one enzyme, wherein the enzyme is selected from the group consisting of: proteases, amylases, lipases, mannanases, cellulases, or mixtures thereof.

2. The agent according to claim 1, wherein the peptide comprises at least: 1 to 5 tryptophan functional groups and/or 1 to 5 arginine functional groups and/or 1 to 5 lysine functional groups; or, at least 1 to 4 tryptophan functional groups and/or 1 to 3 arginine functional groups and/or 1 to 5 lysine functional groups.

3. The agent according to claim 1, wherein the peptide comprises an amino acid sequence that, over the entire length thereof, is at least 70%, and at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5.

4. The agent of claim 1, wherein the agent further comprises;
in each case based on the total weight of the agent,
the at least one peptide in an amount of active peptide of 1×10⁻⁸ to 5 wt. %, 0.0001 to 1 wt. %, 0.0005 to 0.5 wt. %, 0.001 to 0.1 wt. %, the at least one surfactant in an amount of 3 to 35 wt.-%, 5 to 30 wt.-%, based on the total weight of the agent,
the at least one builder in an amount of 0.5 to 50 wt. %, 0.5 to 20 wt. %, 0.5 to 10 wt. %, and
the at least one enzyme in an amount of active protein of 1×10⁻⁸ to 5 wt. %, 0.0001 to 1 wt. %, 0.0005 to 0.5 wt. %, 0.001 to 0.1 wt. %.

5. The agent of claim 1:
wherein the agent comprises, in each case based on the total weight of the agent,
(i) 2 to 20 wt. %, 3 to 17 wt. %, of anionic surfactants,
(ii) 1 to 10 wt. %, 3 to 8 wt. %, of non-ionic surfactants,
(iii) 0 to 1 wt. %, 0 to 0.5 wt. %, of soap, and
(v) 0 to 5 wt. %, 0 to 3 wt. %, of fatty acids.

6. The agent of claim 1:
wherein the builder system comprises, in each case based on the total weight of the agent,
(i) 0 to 10 wt. %, 1 to 4 wt. %, of citric acid and/or citrate, alkali citrate,
(ii) 0 to 40 wt. %, 0 to 15 wt. %, more 1 to 3 wt. %, of alkali carbonate, sodium carbonate,
(iii) 0 to 20 wt. %, 3 to 10 wt. %, of alkali silicate,
(iv) 0 to 10 wt. %, 0.5 to 2 wt. %, of phosphonic acid and/or alkali phosphonate, particularly HEDP and/or DTPMP, and/or
(v) 0 to 10 wt. %, 0.5 to 3 wt. %, of aminopolycarboxylic acids.

7. The agent of claim 1:
wherein,
the agent is substantially free of boron-containing compounds, or free of boron-containing compounds; and/or
the agent is substantially free of phosphonates, or free of phosphonates, in particular free of HEDP and/or DTPMP; and/or
the agent, in a 1 wt. % solution in deionized water at 20° C., has a pH in a range of from about 6 to about 11, in particular from about 6.5 to about 10.5, from about 7 to about 10, from about 8 to about 9; and/or
the agent, based on the total weight of the agent, contains less than 10 wt. % of LAS, less than 6 wt. %, less than 3 wt. %, is substantially free of LAS, the composition is free of LAS; and/or
the agent is substantially free of bleach, or free of bleach.

8. The agent according to claim 1 wherein the agent is used in at least one method step, the method carried out in a temperature range from about 20° C. to about 60° C., about 40° C., or about 30° C.

9. The agent of claim 8, wherein the method step includes contacting the agent to surfaces in or on: households, washing machines, dishwashers, dishes, pump sumps, storage tanks, water reservoirs, washing drums, rinsing chambers, washbasins or sinks, toilet bowls, cisterns, toilet rims, siphons, water pipes and/or lines, water inlets and/or outlets, and/or textiles.

10. The agent of claim 9,
wherein the peptide
has a length of 10 to 50, 10 to 30, 12 to 25, amino acids selected from the group consisting of: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, preferably selected from alanine, arginine, glutamic acid, glycine, isoleucine, leucine, lysine, phenylalanine, proline, serine, threonine, tryptophan, tyrosine, and valine,
has a net charge of +2 to +8, +3 to +7, or +4 to +6, and is antimicrobially active, the antimicrobial activity determined as described in Example 1.

11. (canceled)

12. (canceled)

13. (canceled)

14. The peptide according to claim 10, wherein the peptide comprises at least; 1 to 5 tryptophan functional groups and/or 1 to 5 arginine functional groups and/or 1 to 5 lysine functional groups; at least 1 to 4 tryptophan functional groups and/or 1 to 3 arginine functional groups and/or 1 to 5 lysine functional groups; and/or the peptide comprises an amino acid sequence that, over the entire length thereof, is at least 70% or at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to one of the amino acid sequences set forth in SEQ ID NO:1, SEQ ID NO:2, SEQ ID NO:3, SEQ ID NO:4 or SEQ ID NO:5.

15. The use of a detergent or cleaning agent according claim 1, said use including:
for cleaning textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets, and/or
for reducing bad odors from textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets which have been treated with the agent, and/or
for disinfecting textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets which have been treated with the agent, and/or
for suppressing and/or reducing microbial growth on textiles and/or hard surfaces, in particular dishes, and/or household surfaces, preferably in washing machines or dishwashers, pump sumps, storage tanks, water reservoirs, washing drums, rinsing chambers, washbasins or sinks, toilet bowls, cisterns, toilet rim, siphons, water pipes and/or lines, water inlets and/or outlets which have been treated with the agent,
the use occurring in a temperature range from about 20° C. to about 60° C., about 40° C., or about 30° C.


SEQUENCE LISTING (5 sequences)

SEQ ID NO: 1 - protein, 12 aa, synthetic construct
ILRWPWWPWRRK

SEQ ID NO: 2 - protein, 25 aa, synthetic construct
FSTKTRNWFSEHFKKVKEKLKDTFA

SEQ ID NO: 3 - protein, 22 aa, synthetic construct
KTRNWFSEHFKKVKEKLKDTFA

SEQ ID NO: 4 - protein, 17 aa, synthetic construct
WRWAKWGLKLLKYKKIY

SEQ ID NO: 5 - protein, 17 aa, synthetic construct
IWWAKWGLKLLRYRRWY